SEQUENCE LISTING (33 sequences)

SEQ ID NO: 1 - DNA, 4896 bp, Homo sapiens
gggcagagacacggccgcagggtcctggggctcgctcgctagcctctgcccggcacacatggacttgagcatctggaagacggcgaggggggccacgttcagcttcagcaggtccaccaggatcctggcggggacagacgcggggccggtgagccctctccgcccggcgcggccacctccctgaagacccgacaaggacgggggcgggggctcctcccgaccagcgggccgggcgtacccggaaagcagaggtcggggggtgtctggctagcatgcggcccccacccgccccgctcacttgaacacgtcggggtcgatagcgccgcccgccgcctgcgccagctcgtacagctccatctcctcggtgctcagcaccttcttccgccgcagcgccagcttctgccgggccgcctccagccccgggggcgccgccgaccccggcccaggccctacgccctgcgccgccatccccgcggcccgcggaaaggtgcgctccgccccgccgcgccccctagcggctgcgcccaccaccctctccctgcagcgtcaacgtctggcctcccgggctgccctggcgggaggggcgggagcgcggggacggggccgcctccgcggcgtcattgaagcccaacagttattgggcgctcacggtgtgctgagcggcgctctaggagccgggagtggcatggcggaccgcaggggtccgctgcttggcgatctgggcctccctgatagactgcattggtcgtgctcgcttaggtggcagcacccagcccagtgccaggcatacattgggcgctcggtaaatgcctctttcaagaaagcgtgcaattttctggtccgcaccggtgcaccactaggggtcgcttttcggggcgggcggggggaaggggggggcactaatcaacaatactgcttacgcgcacgcggattccttgctggggagaaagtaccttggggcgccggaggccgccacaacgcaggcgcattcagctaaggaccactccctcccccgcactcctgcctcgccatttctcttccccgcccggccggccttcgctttgcgcacgcgccttttgaggtaacggcccaaagaggtggaagcgcttttcccgcccggccgcggggcgtggctctgcgcgcagcttgatgacgactttcggcgccgtggcggaatggcggcttccatctctgaggcgagcgacgctatggatcccacagtggtttgctaagaaggccattttcaactctccactggaggctgctatggcgttccctcacctgcagcagcccagctttctactggctagcctgaaagctgactctataaataagccctttgcacagcagtgccaagacttggttaaagtcattgaggactttccagcaaaggagctgcacaccatcttcccatggctggtagaaagcatttttggcagcctagatggtgtcctcgttggctggaacctccgctgcttacaggggcgcgtgaatcctgtggagtacagcatcgtgatggaatttctcgaccctggtggcccaatgatgaagttggtttataagcttcaagctgaagactataagttcgactttcctgtctcctacttgcctggtcctgtgaaggcgtccatccaggagtgcatcctccctgacagtcctctgtaccacaacaaggtccagttcacccctactgggggccttggtctgaacttggccctgaatccgttcgagtattacatattcttctttgccttgagcctcatcactcagaagccacttcctgtgtccctccacgtccgtacttcagactgtgcctatttcatcctggtggacaggtacctgtcatggttcctgcccaccgaaggcagtgtgcccccaccactctcctccagcccaggggggaccagcccctcaccacctcccaggacaccagccataccctttgcttcctatggcctccaccacactagcctcctaaagcgacacatctctcatcagacgtctgtgaatgcagaccccgcctcccacgagatctggaggtcagaaactctgctccaggtttttgttgaaatgtggcttcatcactattccttggagatgtatcaaaaaatgcagtcccctcatgccaagctggaggttctgcactaccgactcagtgtctccagcgccctctacagccccgcccaacccagcctccaggccctccacgcctaccaagagtcgttcacgcctactgaggagcatgtgttggtggtgcgcctgctgctgaagcacctgcacgcctttgccaacagcctgaagccagagcaggcctcaccctccgcccactcccacgccaccagccccctggaggagttcaaacgggctgctgtcccgaggttcgtccagcagaaactctacctcttcttgcagcattgctttggccactggcccctggacgcatcgttcagagctgtcctggagatgtggctgagctacctgcagccgtggcggtacgcgcctgacaagcaggctccgggcagcgactcccagccccggtgtgtgtcggagaaatgggcaccctttgtccaggagaacctgctgatgtacaccaagttgtttgtgggctttctgaaccgcgcgctccgcacagacctggtcagccccaagcacgcgctcatggtgttccgagtggccaaagtctttgcccagcccaacctggctgagatgattcagaaaggtgagcagctattcctggagccagagctggtcatcccccaccgccagcaccgactcttcacggcccccacattcactgggagcttcctgtcaccctggccaccagcggtcactgatgcctccttcaaggtgaagagccacgtctacagcctggagggccaggactgcaagtacaccccgatgtttgggcccgaggcccgcaccctggtcctgcgcctcgctcagctcatcacacaggccaaacacacagccaagtccatctccgaccagtgtgcggagagcccggctggccactccttcctctcatggctgggctttagctccatggacaccaatggctcctacacagccaacgacctggacgagatggggcaagacagtgtccggaagacagatgaatacctggagaaggccctggagtacctgcgccagatattccggctcagcgaagcgcagctcaggcagttcacactcgccttgggcaccacccaggatgagaatggaaaaaagcaactccccgactgcatcgtgggtgaggacggactcatccttacgcccctggggcggtaccagatcatcaatgggctgcgaaggtttgaaattgagtaccagggggacccggagctgcagcccatccggagctatgagatcgccagcttggtccgcacactctttaggctgtcgtctgccatcaaccacagatttgcaggacagatggcggctctgtgttcccgggatgacttcctcggcagcttctgtcgctaccacctcacagaacctgggctggccagcaggcacctgctgagccctgtggggcggaggcaggtggccggccacacccgcggccccaggctcagcctgcgcttcctgggcagttaccggacgctggtctcgctgctgctggccttcttcgtggcctctctgttctgcgtcgggcccctcccatgcacgctgctgctcaccctgggctatgtcctctacgcctctgccatgacactgctgaccgagcgggggaagctgcaccagccctgaaggtgtcagctgccttcagagcaggctggagggatttgccacacagccccacccttgggctgagaggacctgggaagcccctccaggagggaacacggtcatcctcgggcttctggagcggggttcctgcagccgcagaggcatctggaggaaacgcaaccaagaaaggaaggcaggtgggccccagcaaaggagtagctgccagggctcaacagctacgctctgtgacagcgcagagctcagcggcggcctttccctccctccgccaaggactcacggccaagccagctctcggggccttttttccactgcccatttggctactctgctgcaccaagcttgggagccagcctgccaacagccacctgggcctggcctccccactggctggccttgaggttggcagagtgggttgtggcgcttcctctctctgtgtgggaccaggacagtggcttaagtctccactccaggaaagaatcaaagtttctagagttgtgagaaaaccagagagtggctgtcctgattcttcactgtgaggggcgttcttcatgttctcccagctgttccaagactgggccgtagaattccatgtttcaggagcctaagaccctcccagagcccaggggcttcaccgcagaccccaagccattgagcacatcacccaaagcagtggccaacatcgcggacccctgtgccttgtcacagatgggtgctggtcctcaggcgttggggacactgctgggtcgatggggtcggattctgccagtttctgctctgcagccaaagatggtcagaagcattgtcacttcagtaacatcaagtgctcaaagacatggcaaccgttcagtggtacttaagtattcaaaatatacaactacagattctctgacagaaaccagcacggggtcttcaccttcattcaccccacaggcgacatgcgagggagaacagcatctcagtggtgatttccaaaccaagcctttgttttcggtgtggggttttgggggtttgctttaatgtttttgaaattgtaaatgttgggctttgtattttgatgtaaactgagcataatggcattttagggcctgtgaccaaaaatgaagcttgtaacgaccatggatctgaataaacatgtccttgcttctg

SEQ ID NO: 2 - DNA, 2598 bp, Homo sapiens
atgacgactttcggcgccgtggcggaatggcggcttccatctctgaggcgagcgacgctatggatcccacagtggtttgctaagaaggccattttcaactctccactggaggctgctatggcgttccctcacctgcagcagcccagctttctactggctagcctgaaagctgactctataaataagccctttgcacagcagtgccaagacttggttaaagtcattgaggactttccagcaaaggagctgcacaccatcttcccatggctggtagaaagcatttttggcagcctagatggtgtcctcgttggctggaacctccgctgcttacaggggcgcgtgaatcctgtggagtacagcatcgtgatggaatttctcgaccctggtggcccaatgatgaagttggtttataagcttcaagctgaagactataagttcgactttcctgtctcctacttgcctggtcctgtgaaggcgtccatccaggagtgcatcctccctgacagtcctctgtaccacaacaaggtccagttcacccctactgggggccttggtctgaacttggccctgaatccgttcgagtattacatattcttctttgccttgagcctcatcactcagaagccacttcctgtgtccctccacgtccgtacttcagactgtgcctatttcatcctggtggacaggtacctgtcatggttcctgcccaccgaaggcagtgtgcccccaccactctcctccagcccaggggggaccagcccctcaccacctcccaggacaccagccataccctttgcttcctatggcctccaccacactagcctcctaaagcgacacatctctcatcagacgtctgtgaatgcagaccccgcctcccacgagatctggaggtcagaaactctgctccaggtttttgttgaaatgtggcttcatcactattccttggagatgtatcaaaaaatgcagtcccctcatgccaagctggaggttctgcactaccgactcagtgtctccagcgccctctacagccccgcccaacccagcctccaggccctccacgcctaccaagagtcgttcacgcctactgaggagcatgtgttggtggtgcgcctgctgctgaagcacctgcacgcctttgccaacagcctgaagccagagcaggcctcaccctccgcccactcccacgccaccagccccctggaggagttcaaacgggctgctgtcccgaggttcgtccagcagaaactctacctcttcttgcagcattgctttggccactggcccctggacgcatcgttcagagctgtcctggagatgtggctgagctacctgcagccgtggcggtacgcgcctgacaagcaggctccgggcagcgactcccagccccggtgtgtgtcggagaaatgggcaccctttgtccaggagaacctgctgatgtacaccaagttgtttgtgggctttctgaaccgcgcgctccgcacagacctggtcagccccaagcacgcgctcatggtgttccgagtggccaaagtctttgcccagcccaacctggctgagatgattcagaaaggtgagcagctattcctggagccagagctggtcatcccccaccgccagcaccgactcttcacggcccccacattcactgggagcttcctgtcaccctggccaccagcggtcactgatgcctccttcaaggtgaagagccacgtctacagcctggagggccaggactgcaagtacaccccgatgtttgggcccgaggcccgcaccctggtcctgcgcctcgctcagctcatcacacaggccaaacacacagccaagtccatctccgaccagtgtgcggagagcccggctggccactccttcctctcatggctgggctttagctccatggacaccaatggctcctacacagccaacgacctggacgagatggggcaagacagtgtccggaagacagatgaatacctggagaaggccctggagtacctgcgccagatattccggctcagcgaagcgcagctcaggcagttcacactcgccttgggcaccacccaggatgagaatggaaaaaagcaactccccgactgcatcgtgggtgaggacggactcatccttacgcccctggggcggtaccagatcatcaatgggctgcgaaggtttgaaattgagtaccagggggacccggagctgcagcccatccggagctatgagatcgccagcttggtccgcacactctttaggctgtcgtctgccatcaaccacagatttgcaggacagatggcggctctgtgttcccgggatgacttcctcggcagcttctgtcgctaccacctcacagaacctgggctggccagcaggcacctgctgagccctgtggggcggaggcaggtggccggccacacccgcggccccaggctcagcctgcgcttcctgggcagttaccggacgctggtctcgctgctgctggccttcttcgtggcctctctgttctgcgtcgggcccctcccatgcacgctgctgctcaccctgggctatgtcctctacgcctctgccatgacactgctgaccgagcgggggaagctgcaccagccc

SEQ ID NO: 3 - protein, 866 aa, Homo sapiens
MTTFGAVAEWRLPSLRRATLWIPQWFAKKAIFNSPLEAAMAFPHLQQPSFLLASLKADSINKPFAQQCQDLVKVIEDFPAKELHTIFPWLVESIFGSLDGVLVGWNLRCLQGRVNPVEYSIVMEFLDPGGPMMKLVYKLQAEDYKFDFPVSYLPGPVKASIQECILPDSPLYHNKVQFTPTGGLGLNLALNPFEYYIFFFALSLITQKPLPVSLHVRTSDCAYFILVDRYLSWFLPTEGSVPPPLSSSPGGTSPSPPPRTPAIPFASYGLHHTSLLKRHISHQTSVNADPASHEIWRSETLLQVFVEMWLHHYSLEMYQKMQSPHAKLEVLHYRLSVSSALYSPAQPSLQALHAYQESFTPTEEHVLVVRLLLKHLHAFANSLKPEQASPSAHSHATSPLEEFKRAAVPRFVQQKLYLFLQHCFGHWPLDASFRAVLEMWLSYLQPWRYAPDKQAPGSDSQPRCVSEKWAPFVQENLLMYTKLFVGFLNRALRTDLVSPKHALMVFRVAKVFAQPNLAEMIQKGEQLFLEPELVIPHRQHRLFTAPTFTGSFLSPWPPAVTDASFKVKSHVYSLEGQDCKYTPMFGPEARTLVLRLAQLITQAKHTAKSISDQCAESPAGHSFLSWLGFSSMDTNGSYTANDLDEMGQDSVRKTDEYLEKALEYLRQIFRLSEAQLRQFTLALGTTQDENGKKQLPDCIVGEDGLILTPLGRYQIINGLRRFEIEYQGDPELQPIRSYEIASLVRTLFRLSSAINHRFAGQMAALCSRDDFLGSFCRYHLTEPGLASRHLLSPVGRRQVAGHTRGPRLSLRFLGSYRTLVSLLLAFFVASLFCVGPLPCTLLLTLGYVLYASAMTLLTERGKLHQP

SEQ ID NO: 4 - protein, 827 aa, Canis familiaris
MAFPHLQQPSFLLASLKADSINKPFAQRCQDLVKVIEDFPAKELHTIFPWLVESIFGSLDGVLVGWNLRCLQGRVNPMEYSIAMEFLDPGGPMMKLVYKLQAEDYKFDFPVSYLPGPVKASIQERVLPDSPLYHNKVQFPATGGLGLNLALNPFEYYMFFFALSLITQKPLPGALHIRTSDCAYFILVDRYLSWFLPTEGSVLPPLSSSPGGPSPSPAPRTPAIPFASYGLHHTSLLKRHVSHQTSVNADPASHEIWRSETLLQVFVEMWLHHYSLEMYQKMQSPHAKLEVLHYRLSVSSALHSPAQPSLQALHAYQESFTPTEEHVLVVRLLLKHLHAFSNSLKPEQVSPSAHSHATSPLEEFKRAAVPRFVQQKLYLFLQHCFGHWPLDASFRAVLEMWLSYLQPWRYAPEKPAPSSDCQARCVSERWAPFVQENLLMYTKLFVGFLSRALRTDLVSPKNALMVFRVAKVFAQPNLAEMIQKGEQLFLEPELVIPHRQHRLFTAPTFTGSFLSSWPPAVTDTSFRVKSHVYSLEGQDCKYTPMFGPEVRTLVLRLAQLITQAKQTAKSISDQCGESTPGHPFLSWLGFCSTDTNGSYPANDLDEMGQDSVRKTDEYLEKALEYLCQMFRLSEAQLAQLTLALGTTQDENGKKQLPDCIVGEDGLILTPLGRYQIINGLRKFDIEYQGDLELQPIRSYEIASLVRMLFRLSSAINCRFAGQMAALCSRADFLGSFCRYHLTEPGVTGRHLLSPVARESAASRARGPRLSLRFLGSYRTLLSLLLAFFVASLFCIGPLPCALLLMLGYLLYAVAMTLLTERSKLHQL

SEQ ID NO: 5 - protein, 823 aa, Mus musculus
MAFPHLQQPSFLLASLKADSINKPFAQRCQDLVKVIEDFPAKELHAVFPWLVESIFGSLDGVLVGWNLRCLQGRVNPVEYSTAMEFLDPSGPMMKLVYKLQAEDYNFDFPVSCLPGPVKASIQENVLPDSPLYHNKVQFPPTGGLGLNLALNPFEYYMFYFALSLISQKPMSMTLHVRTSDCAYFTLVDRYLSWFLPTEGSVPPPLCSSPGGSSPSPAPRTPAMPFASYGLHTSLLKRHISHQTSVNADPASHEIWRSETLLQVFVEMWLHHYSLEMYQKMQSPHAKLEVLHYRLTVSSALHSPAQPSLQALHAYQESFTPTEEHVLVVRLLLKHLHAFANSLKPDQASPSAHSHATSPLEEFKRAAEPRFVQQKLYVFLQHCFGHWPLDATFRAVLEMWLSYLQPWRYAPEKQAQGSDPQPRCVSEKWAPFIQENLLMYTKLFVSFLNRALRTDLVSPKNALMVFRVAKVFAQPNLAEMIQKGEQLFLEPELIIPHRQHRLFTVTTSFLSPWPPVVTDASFKVKSHVYSLEGQDCKYTPMFGPEIRTLVLRLAQLITQAKQTAKSISDQYVESPTGRSFLSWLTFGLTDTNSCYPANDLDEIGQDSIRKTDEYLEKALEYLRQIFRLSEAQLAQLTLALGSARDENGKQQLPDCIVGEEGLILTPLGRYQIINGLRRFEIEYQGDLELQPIRSYEITSLVRALFRLSSAINRRFAGQMAALCSRNDFLGSFCRYHLTEPALSNRHLLSPVGRRQVTNPARGPRLSLRFLGSYRTLLLLLMAFFVASLFCIGPLSCSLLLVLGYVLYAIAMTLLTERGKLHQL

SEQ ID NO: 6 - protein, 851 aa, Gallus gallus
MNDDANESATAEARREARLGDMAGPYLQQPSFLLATLKADCVNKPFAQRCHDLETVIEEFPAKELHGIFPWLVESIFGSLDGTIVGWNLRCLQGRTNPTEYSVALDFLDPSGPMMKLVYKLQAEEYRYDFPVSYLPGPVKASIQEQVLPECSLYHNKVQFPSSGGLGLNLALNPFEYYMFYFAISLITQKNSPITHHISPSNSAYFILVDTYLKCFLPTEGSVLPPPSSNPGGAIPSPTPRSPAVPFTSYGMHHTSLLKRHIAHQPSVNADPASQEIWRSETLLQIFVEMWLHHYSLEMYQKMQSPHIKLEVLHYRLSISSKHHGSPAQSSYQALHAYQESFKPTEEHVLVVRLLVKHLHAFSNSLKPEPLSPSAHSHTASPLEEFKRVVIPRFVQQKLYIFLQHCFGHWPLDASFRAVLEMWLSYLQPWRYAPEKPPQTAELLPRSVSEKWAAFIQENLLMYTKLFVGFLNRTLRTDLVSPKNALMVFRVAKVFAQPNLAEMILKGEQLFLEPELVIPHRQHRLFMTPTLGGSFLSSWPPAITDASFKVKSHVYSLEGQDFQYKQMFGTEVRNLVLKLAQLICQAQQTAKSISDHSAETTASHSFFSWFKFTPSEMNGSYTGNDLDEIGQDSIKKTDEYLEKALEYLCQIFKLNEAQLTQMMMKCGTAQDENGKKQLPDCIESENGLILTPLGRYQMINGLRRFDVQYQGDTELQPIRSYENATLVRLLYRLSSALNERLADQMDVLCSREDFVGRFCRYHLTNPHLVHKIRYSPVLKDRTSQNWGPRISLRFLASYRTLISLLMLYFIASWFHIGPRLHSFQKLSLFMPNHFSANQKPVPLIFDKVPKH

SEQ ID NO: 7 - protein, 791 aa, Danio rerio
MAASALQQPSYLLANLKADWTNKPLHQRCHELCKIIDDYPAKELHAIFPWLVECVFGSLDGILTGWNLRFLQARSAEYSIAMEFLDPSGPMMKLVYKLQAEEYKYEFPISYLPGPIKSSIHAGVLPDCPLFHNKIQFPMSGLLFLNPFEYYMFNFASSLIAPKNYPQGQHGSSSDSAYFVLVDTYLKYFLPTEGNVPPSPFSDTRGTVASPAPRSTNVPYVGYGGHSTSLLKRHITHQSSVNADPAAQEIWRSETLLQVFVEMWLHHYSLEMYQKLQSPQVKEPFMPSEEHVLVVRLLVKHLHTFSSSLKPESISSSPSAHSHSSPLEELKRVVVQRFVQQKLYVFLQHCFGHWPLDASFRAVLETWLSYIQPWRYTGDKNNTQTDGPNRTVPDKWASFVQENLLLYTKLFQGFLNRAMRTDLVNAKNALMVFRVAKVFAQPSLSEMIQKGEQLFLEPEHAILQRHNRVFLTPSHGGSFLSARQPMGTDNVFKVKSHVYSLEGQDCQYNLMFGPDQRKNVLKLIQIIAQARQTAKRISDHSTEMAANNSFLSWFGVGSPDHNSTFTGGEMDEMGGEGVKKTHEFLDKALDYLCQIFRLNAGQLSQLISNVASVDNNGASKQLPDCIPSENGLVLTDLGRLQIINGLRRFEIEYQGDPELQPIRSYENAFLVRLLFQISSFINERLGEHMEVLCSRQDFLGSVGRHYLSSSSAVVEQRRKSPVTRQMRDRPQRARLSLRALASYRTLLTLLLLYMLFALLSFGLFSSTGLILIISFLYELLSNFFHEKLKTH

SEQ ID NO: 8 - protein, 767 aa, Drosophila melanogaster
MSQTMPPENLSSRLLTVLSLPLFERVHELSILFDRCSLRQVQEIFPHVVHSIFGIDGNPLGWGLRTTTLENNPVQFQTLHQFFGVCGPLMHVCHRLLVDQYKFELDINLLPAKFVSLLQNGQNPSFYAELINVETMMHQVSTLSLNAFDFYVIHFVLYALQPLHSINPIAMQIHNVRSKTVYLKLVAEYLNNFLPLLPDARIEPVHFSSGVKAPQPLPAQALQPQRQPRYIKIPSSYRNGGNVSGGGGIPSASVGVGNISPQSNSPSASSNRAYAWRSESVLHFFVDIWLRYDIESEHHLPSSDFVRGVRTLVKQVHFFANGAQHDHSSLCALRKVSLSMVKARIYAFIVGLIDRWPLDSSLMVVLELWLSYIQPWRYTIASLNNKTPSLSYRPPISSCFDGFIIDNLIMYTHIFMQLLPHFGRLDYTVYRNAFMLYRLATIFSQHDLVDKLQRFEAIHAGNSFGFDSPQRQVNIMNKSVSPGTPWNQAVNISSAKLFSYTMQTQMETFLLLISMARNSVLRDIAKLRNEIAERQRSEGFLKNFYKKIFGECTQEEVTLREFSRIPEVLRQCIDAFCRTFNVDQANLSMHETLPEEPSVPSAPAVQNFSFFDASDTLDTSKLNPHQMSLNASSLHAAVDPATLPIQTNEVRALVRMLHFVSEKINNKYGSQLQGFYVRDDYFGKVARQLLYAPMTEQWFDKSSGHVEICENLVPPRVCLRPLGSIPALTTIGCSMIFGQLVWGAPLLGIFILATVLLVYIMLQALFS

SEQ ID NO: 9 - protein, 640 aa, Caenorhabditis elegans
MFQQRQTPTEFSQMDDVVSICREISSNIISTDKNVPISKMIIHKIFKNPHIDLCSILPPLIQQHYPDSPYCHIQSLINSNSVFFETVLEDDQLCTELWDSILHQCEVSTAPTEPAKDSILFSAVSQYVTRILPCDETAPSSMSVPRSPKITHQTIRTPLQLFRADSPLVNRVSIEERSQMMSVSTVSFQPRLVQNFCNFVTKSCTTAQFTNANKLLLIRYLLKQFHVFCQFSTGDPEVDNWKLDLMASAPFRFSLYDFFKSSVTQVPTTIVFRDITMCWLTYCRPWRYHNMSNPSDFAPAAKYRQFFEKNVEFYEVILGKILKRFSAFEMCEELLGSLRAIIEFAWKEPQTLLLRHIQLDIQPHSLELLKQMQVVVRMHRSAIQKEQDEHSGFWNSLFYSGDSARTQSSRRLVSELLTLMKDSDSYVGTHFMPEMEDSVLANRTGNETFHKEKLYTSLLNETPKSGLGIPDHFVDGPSNHMLLTPIGQRQVLNREKRFDYSNCIKDDPKAPTRSYELGIAVKLTTSLAQKLNELSFVQKIGDHYSQQSVIGAIARKVMYPPVPNLDPNATVPAYSARIIRSPPLLRLRYFASYQSIFFMIWLLFALKYGLTFTIVSLILIALSVTVLLFIELDSGSSSSY

SEQ ID NO: 10 - DNA, 7303 bp, Artificial
  pRK-nSMase3
tatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcgttagaacgcggctacaattaatacataaccttatgtatcatacacatacgatttaggtgacactatagaataacatccactttgcctttctctccacaggtgtccactcccaggtccaactgcacctcggttctatcgattgaattctgatgacgactttcggcgccgtggcggaatggcggcttccatctctgaggcgagcgacgctatggatcccacagtggtttgctaagaaggccattttcaactctccactggaggctgctatggcgttccctcacctgcagcagcccagctttctactggctagcctgaaagctgactctataaataagccctttgcacagcagtgccaagacttggttaaagtcattgaggactttccagcaaaggagctgcacaccatcttcccatggctggtagaaagcatttttggcagcctagatggtgtcctcgttggctggaacctccgctgcttacaggggcgcgtgaatcctgtggagtacagcatcgtgatggaatttctcgaccctggtggcccaatgatgaagttggtttataagcttcaagctgaagactataagttcgactttcctgtctcctacttgcctggtcctgtgaaggcgtccatccaggagtgcatcctccctgacagtcctctgtaccacaacaaggtccagttcacccctactgggggccttggtctgaacttggccctgaatccgttcgagtattacatattcttctttgccttgagcctcatcactcagaagccacttcctgtgtccctccacgtccgtacttcagactgtgcctatttcatcctggtggacaggtacctgtcatggttcctgcccaccgaaggcagtgtgcccccaccactctcctccagcccaggggggaccagcccctcaccacctcccaggacaccagccataccctttgcttcctatggcctccaccacactagcctcctaaagcgacacatctctcatcagacgtctgtgaatgcagaccccgcctcccacgagatctggaggtcagaaactctgctccaggtttttgttgaaatgtggcttcatcactattccttggagatgtatcaaaaaatgcagtcccctcatgccaagctggaggttctgcactaccgactcagtgtctccagcgccctctacagccccgcccaacccagcctccaggccctccacgcctaccaagagtcgttcacgcctactgaggagcatgtgttggtggtgcgcctgctgctgaagcacctgcacgcctttgccaacagcctgaagccagagcaggcctcaccctccgcccactcccacgccaccagccccctggaggagttcaaacgggctgctgtcccgaggttcgtccagcagaaactctacctcttcttgcagcattgctttggccactggcccctggacgcatcgttcagagctgtcctggagatgtggctgagctacctgcagccgtggcggtacgcgcctgacaagcaggctccgggcagcgactcccagccccggtgtgtgtcggagaaatgggcaccctttgtccaggagaacctgctgatgtacaccaagttgtttgtgggctttctgaaccgcgcgctccgcacagacctggtcagccccaagcacgcgctcatggtgttccgagtggccaaagtctttgcccagcccaacctggctgagatgattcagaaaggtgagcagctattcctggagccagagctggtcatcccccaccgccagcaccgactcttcacggcccccacattcactgggagcttcctgtcaccctggccaccagcggtcactgatgcctccttcaaggtgaagagccacgtctacagcctggagggccaggactgcaagtacaccccgatgtttgggcccgaggcccgcaccctggtcctgcgcctcgctcagctcatcacacaggccaaacacacagccaagtccatctccgaccagtgtgcggagagcccggctggccactccttcctctcatggctgggctttagctccatggacaccaatggctcctacacagccaacgacctggacgagatggggcaagacagtgtccggaagacagatgaatacctggagaaggccctggagtacctgcgccagatattccggctcagcgaagcgcagctcaggcagttcacactcgccttgggcaccacccaggatgagaatggaaaaaagcaactccccgactgcatcgtgggtgaggacggactcatccttacgcccctggggcggtaccagatcatcaatgggctgcgaaggtttgaaattgagtaccagggggacccggagctgcagcccatccggagctatgagatcgccagcttggtccgcacactctttaggctgtcgtctgccatcaaccacagatttgcaggacagatggcggctctgtgttcccgggatgacttcctcggcagcttctgtcgctaccacctcacagaacctgggctggccagcaggcacctgctgagccctgtggggcggaggcaggtggccggccacacccgcggccccaggctcagcctgcgcttcctgggcagttaccggacgctggtctcgctgctgctggccttcttcgtggcctctctgttctgcgtcgggcccctcccatgcacgctgctgctcaccctgggctatgtcctctacgcctctgccatgacactgctgaccgagcgggggaagctgcaccagccctgacgtcgacctgcagaagcttggccgccatggcccaacttgtttattgcagcttataatggttacaaataaagcaatagcatcacaaatttcacaaataaagcatttttttcactgcattctagttgtggtttgtccaaactcatcaatgtatcttatcatgtctggatcgatcgggaattaattcggcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaactatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgttaacagcttggcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgtagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccaactccgctatcgctacgtgactgggtcatggctgcgccccgacacccgccaacacccgctgacgcgccctgccgggcttgtctgctcccggcatccgcttacagacaagctgtgaccgtctccgggagctgcatgtgtcagaggttttcaccgtcatcaccgaaacgcgcgaggcagtattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgatgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgccagcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataacgcgcagcggtcgggctgaacggggggttcgtgcacacagcccagcttggagcgaacgacctacaccgaactgagatacctacagcctgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgaatcattaatccagctggcacgacaggtttcccgactggaaagcgggcagtgagcgcaacgcaattaatgtgagttacctcactcattaggcaccccaggctttacactttatgcttccggctcgtatgttgtgtggaattgtgagcggataacaatttcacacaggaaacagctatgaccatgattacgaattaattcgagctcgcccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatca

SEQ ID NO: 11 - DNA, 6678 bp, Artificial
  pRK-nSMase2
tatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcgttagaacgcggctacaattaatacataaccttatgtatcatacacatacgatttaggtgacactatagaataacatccactttgcctttctctccacaggtgtccactcccaggtccaactgcacctcggttctatcgattgaattccccggggatccatggttttgtacacgaccccctttcctaacagctgtctgtccgccctgcactgtgtgtcctgggcccttatctttccatgctactggctggtggaccggctcgctgcctccttcatacccaccacctacgagaagcgccagcgggcagacgacccgtgctgcctgcagctgctctgcactgccctcttcacgcccatctacctggccctcctggtggcctcgctgccctttgcgtttctcggctttctcttctggtccccactgcagtcggcccgccggccctacatctattcacggctggaagacaagggcctggccggtggggcagccctgctcagtgaatggaagggcacggggcctggcaaaagcttctgctttgccactgccaacgtctgcctcctgcccgactcactcgccagggtcaacaacctttttaacacccaagcgcgggccaaggagatcgggcagagaatccgcaatggggccgcccggccccagatcaaaatttacatcgactcccccaccaatacctccatcagcgccgctagcttcagcagcctggtgtcaccacagggcggcgatggggtggcccgggccgtccccgggagcattaagaggacagcctctgtggagtacaagggtgacggtgggcggcaccccggtgacgaggctgccaacggcccagcctctggggaccctgtcgacagcagcagcccggaggatgcctgcatcgtgcgcatcggtggcgaggagggcggccggccacctgaagctgacgaccctgtgcctgggggccaggccaggaacggagctggcgggggcccaaggggccagacgcccaaccataatcagcaggacggggattcagggagcctgggcagcccctcggcctcccgggagtccctggtgaaggggcgagctgggccagacaccagtgccagcggggagccaggtgccaacagcaagctcctgtacaaggcctcggtggtgaagaaggcggctgcacgcaggaggcggcaccccgacgaggccttcgaccatgaggtctccgccttcttccccgccaacctggacttcctgtgcctgcaggaggtgtttgacaagcgagcagccaccaaattgaaagagcagctgcacggctacttcgagtacatcctgtacgacgtcggggtctacggctgccagggctgctgcagcttcaagtgtctcaacagcggcctcctctttgccagccgctaccccatcatggacgtggcctatcactgttaccccaacaagtgtaacgacgatgccctggcctctaagggagctctgtttctcaaggtgcaggtgggaagcacacctcaggaccaaagaatcgtcgggtacatcgcctgcacacacctgcatgccccacaagaggacagcgccatccggtgtgggcagctggacctgcttcaggactggctggctgatttccgaaaatctacctcctcgtccagcgcagccaaccccgaggagctggtggcatttgacgtcgtctgtggagatttcaactttgataactgctcctctgacgacaagctggagcagcaacactccctgttcacccactacagggacccctgccgcctggggcctggtgaggagaagccgtgggccatcggtactctgctggacacgaacggcctgtacgatgaggatgtgtgcacccccgacaacctgcagaaggtcctggagagtgaggagggccgcagggagtacctggcgtttcccaccagcaagagctcgggccagaaggggcggaaggagctgctgaagggcaacggccggcgcatcgactacatgctgcatgcagaggaggggctgtgcccagactggaaggccgaggtggaagaattcagttttatcacccagctgtccggcctgacggaccacctgccagtagccatgcgactgatggtgtcttcgggggaggaggaggcatagctcgacctgcagaagcttggccgccatggcccaacttgtttattgcagcttataatggttacaaataaagcaatagcatcacaaatttcacaaataaagcatttttttcactgcattctagttgtggtttgtccaaactcatcaatgtatcttatcatgtctggatcgatcgggaattaattcggcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaactatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgttaacagcttggcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgtagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccaactccgctatcgctacgtgactgggtcatggctgcgccccgacacccgccaacacccgctgacgcgccctgccgggcttgtctgctcccggcatccgcttacagacaagctgtgaccgtctccgggagctgcatgtgtcagaggttttcaccgtcatcaccgaaacgcgcgaggcagtattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgatgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgccagcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataacgcgcagcggtcgggctgaacggggggttcgtgcacacagcccagcttggagcgaacgacctacaccgaactgagatacctacagcctgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgaatcattaatccagctggcacgacaggtttcccgactggaaagcgggcagtgagcgcaacgcaattaatgtgagttacctcactcattaggcaccccaggctttacactttatgcttccggctcgtatgttgtgtggaattgtgagcggataacaatttcacacaggaaacagctatgaccatgattacgaattaattcgagctcgcccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatca

SEQ ID NO: 12 - DNA, 8433 bp, Artificial
  pYES-nSMase3
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttggtaccgagctcggatccactagtaacggccgccagtgtgctggaattctgatgacgactttcggcgccgtggcggaatggcggcttccatctctgaggcgagcgacgctatggatcccacagtggtttgctaagaaggccattttcaactctccactggaggctgctatggcgttccctcacctgcagcagcccagctttctactggctagcctgaaagctgactctataaataagccctttgcacagcagtgccaagacttggttaaagtcattgaggactttccagcaaaggagctgcacaccatcttcccatggctggtagaaagcatttttggcagcctagatggtgtcctcgttggctggaacctccgctgcttacaggggcgcgtgaatcctgtggagtacagcatcgtgatggaatttctcgaccctggtggcccaatgatgaagttggtttataagcttcaagctgaagactataagttcgactttcctgtctcctacttgcctggtcctgtgaaggcgtccatccaggagtgcatcctccctgacagtcctctgtaccacaacaaggtccagttcacccctactgggggccttggtctgaacttggccctgaatccgttcgagtattacatattcttctttgccttgagcctcatcactcagaagccacttcctgtgtccctccacgtccgtacttcagactgtgcctatttcatcctggtggacaggtacctgtcatggttcctgcccaccgaaggcagtgtgcccccaccactctcctccagcccaggggggaccagcccctcaccacctcccaggacaccagccataccctttgcttcctatggcctccaccacactagcctcctaaagcgacacatctctcatcagacgtctgtgaatgcagaccccgcctcccacgagatctggaggtcagaaactctgctccaggtttttgttgaaatgtggcttcatcactattccttggagatgtatcaaaaaatgcagtcccctcatgccaagctggaggttctgcactaccgactcagtgtctccagcgccctctacagccccgcccaacccagcctccaggccctccacgcctaccaagagtcgttcacgcctactgaggagcatgtgttggtggtgcgcctgctgctgaagcacctgcacgcctttgccaacagcctgaagccagagcaggcctcaccctccgcccactcccacgccaccagccccctggaggagttcaaacgggctgctgtcccgaggttcgtccagcagaaactctacctcttcttgcagcattgctttggccactggcccctggacgcatcgttcagagctgtcctggagatgtggctgagctacctgcagccgtggcggtacgcgcctgacaagcaggctccgggcagcgactcccagccccggtgtgtgtcggagaaatgggcaccctttgtccaggagaacctgctgatgtacaccaagttgtttgtgggctttctgaaccgcgcgctccgcacagacctggtcagccccaagcacgcgctcatggtgttccgagtggccaaagtctttgcccagcccaacctggctgagatgattcagaaaggtgagcagctattcctggagccagagctggtcatcccccaccgccagcaccgactcttcacggcccccacattcactgggagcttcctgtcaccctggccaccagcggtcactgatgcctccttcaaggtgaagagccacgtctacagcctggagggccaggactgcaagtacaccccgatgtttgggcccgaggcccgcaccctggtcctgcgcctcgctcagctcatcacacaggccaaacacacagccaagtccatctccgaccagtgtgcggagagcccggctggccactccttcctctcatggctgggctttagctccatggacaccaatggctcctacacagccaacgacctggacgagatggggcaagacagtgtccggaagacagatgaatacctggagaaggccctggagtacctgcgccagatattccggctcagcgaagcgcagctcaggcagttcacactcgccttgggcaccacccaggatgagaatggaaaaaagcaactccccgactgcatcgtgggtgaggacggactcatccttacgcccctggggcggtaccagatcatcaatgggctgcgaaggtttgaaattgagtaccagggggacccggagctgcagcccatccggagctatgagatcgccagcttggtccgcacactctttaggctgtcgtctgccatcaaccacagatttgcaggacagatggcggctctgtgttcccgggatgacttcctcggcagcttctgtcgctaccacctcacagaacctgggctggccagcaggcacctgctgagccctgtggggcggaggcaggtggccggccacacccgcggccccaggctcagcctgcgcttcctgggcagttaccggacgctggtctcgctgctgctggccttcttcgtggcctctctgttctgcgtcgggcccctcccatgcacgctgctgctcaccctgggctatgtcctctacgcctctgccatgacactgctgaccgagcgggggaagctgcaccagccctgacgtcgagcatgcatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccattcagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 13 - DNA, 7766 bp, Artificial
  pYES-nSMase2
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttggtaccgagctcggatccatggttttgtacacgaccccctttcctaacagctgtctgtccgccctgcactgtgtgtcctgggcccttatctttccatgctactggctggtggaccggctcgctgcctccttcatacccaccacctacgagaagcgccagcgggcagacgacccgtgctgcctgcagctgctctgcactgccctcttcacgcccatctacctggccctcctggtggcctcgctgccctttgcgtttctcggctttctcttctggtccccactgcagtcggcccgccggccctacatctattcacggctggaagacaagggcctggccggtggggcagccctgctcagtgaatggaagggcacggggcctggcaaaagcttctgctttgccactgccaacgtctgcctcctgcccgactcactcgccagggtcaacaacctttttaacacccaagcgcgggccaaggagatcgggcagagaatccgcaatggggccgcccggccccagatcaaaatttacatcgactcccccaccaatacctccatcagcgccgctagcttcagcagcctggtgtcaccacagggcggcgatggggtggcccgggccgtccccgggagcattaagaggacagcctctgtggagtacaagggtgacggtgggcggcaccccggtgacgaggctgccaacggcccagcctctggggaccctgtcgacagcagcagcccggaggatgcctgcatcgtgcgcatcggtggcgaggagggcggccggccacctgaagctgacgaccctgtgcctgggggccaggccaggaacggagctggcgggggcccaaggggccagacgcccaaccataatcagcaggacggggattcagggagcctgggcagcccctcggcctcccgggagtccctggtgaaggggcgagctgggccagacaccagtgccagcggggagccaggtgccaacagcaagctcctgtacaaggcctcggtggtgaagaaggcggctgcacgcaggaggcggcaccccgacgaggccttcgaccatgaggtctccgccttcttccccgccaacctggacttcctgtgcctgcaggaggtgtttgacaagcgagcagccaccaaattgaaagagcagctgcacggctacttcgagtacatcctgtacgacgtcggggtctacggctgccagggctgctgcagcttcaagtgtctcaacagcggcctcctctttgccagccgctaccccatcatggacgtggcctatcactgttaccccaacaagtgtaacgacgatgccctggcctctaagggagctctgtttctcaaggtgcaggtgggaagcacacctcaggaccaaagaatcgtcgggtacatcgcctgcacacacctgcatgccccacaagaggacagcgccatccggtgtgggcagctggacctgcttcaggactggctggctgatttccgaaaatctacctcctcgtccagcgcagccaaccccgaggagctggtggcatttgacgtcgtctgtggagatttcaactttgataactgctcctctgacgacaagctggagcagcaacactccctgttcacccactacagggacccctgccgcctggggcctggtgaggagaagccgtgggccatcggtactctgctggacacgaacggcctgtacgatgaggatgtgtgcacccccgacaacctgcagaaggtcctggagagtgaggagggccgcagggagtacctggcgtttcccaccagcaagagctcgggccagaaggggcggaaggagctgctgaagggcaacggccggcgcatcgactacatgctgcatgcagaggaggggctgtgcccagactggaaggccgaggtggaagaattcagttttatcacccagctgtccggcctgacggaccacctgccagtagccatgcgactgatggtgtcttcgggggaggaggaggcatagctcgagcatgcatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccattcagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 14 - DNA, 5487 bp, Artificial
  AdCGI; bicistronic adenoviral vector encoding EGFP
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggataggatcaagcttatcgataccgtcgacctcgagctcaagcttcgaattctgcagtcgacggtaccgcgggcccgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcatcgatccataacttcgtataatgtatgctatacgaagttatccagatctggttctatagtgtcacctaaatcgtatgtgtatgatacataaggttatgtattaattgtagccgcgttctaacgacaatatgtccatagggcccctacgtcacccgccccgttcccacgccccgcgccacgtcacaaactccaccccctcattatcatattggcttcaatccaaaataaggtatattattgatgatggccgcagcggcccctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggggccgctgcggc

SEQ ID NO: 15 - DNA, 7318 bp, Artificial
  pGFP-nSMase3
tatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtactcagatctcgagatgacgactttcggcgccgtggcggaatggcggcttccatctctgaggcgagcgacgctatggatcccacagtggtttgctaagaaggccattttcaactctccactggaggctgctatggcgttccctcacctgcagcagcccagctttctactggctagcctgaaagctgactctataaataagccctttgcacagcagtgccaagacttggttaaagtcattgaggactttccagcaaaggagctgcacaccatcttcccatggctggtagaaagcatttttggcagcctagatggtgtcctcgttggctggaacctccgctgcttacaggggcgcgtgaatcctgtggagtacagcatcgtgatggaatttctcgaccctggtggcccaatgatgaagttggtttataagcttcaagctgaagactataagttcgactttcctgtctcctacttgcctggtcctgtgaaggcgtccatccaggagtgcatcctccctgacagtcctctgtaccacaacaaggtccagttcacccctactgggggccttggtctgaacttggccctgaatccgttcgagtattacatattcttctttgccttgagcctcatcactcagaagccacttcctgtgtccctccacgtccgtacttcagactgtgcctatttcatcctggtggacaggtacctgtcatggttcctgcccaccgaaggcagtgtgcccccaccactctcctccagcccaggggggaccagcccctcaccacctcccaggacaccagccataccctttgcttcctatggcctccaccacactagcctcctaaagcgacacatctctcatcagacgtctgtgaatgcagaccccgcctcccacgagatctggaggtcagaaactctgctccaggtttttgttgaaatgtggcttcatcactattccttggagatgtatcaaaaaatgcagtcccctcatgccaagctggaggttctgcactaccgactcagtgtctccagcgccctctacagccccgcccaacccagcctccaggccctccacgcctaccaagagtcgttcacgcctactgaggagcatgtgttggtggtgcgcctgctgctgaagcacctgcacgcctttgccaacagcctgaagccagagcaggcctcaccctccgcccactcccacgccaccagccccctggaggagttcaaacgggctgctgtcccgaggttcgtccagcagaaactctacctcttcttgcagcattgctttggccactggcccctggacgcatcgttcagagctgtcctggagatgtggctgagctacctgcagccgtggcggtacgcgcctgacaagcaggctccgggcagcgactcccagccccggtgtgtgtcggagaaatgggcaccctttgtccaggagaacctgctgatgtacaccaagttgtttgtgggctttctgaaccgcgcgctccgcacagacctggtcagccccaagcacgcgctcatggtgttccgagtggccaaagtctttgcccagcccaacctggctgagatgattcagaaaggtgagcagctattcctggagccagagctggtcatcccccaccgccagcaccgactcttcacggcccccacattcactgggagcttcctgtcaccctggccaccagcggtcactgatgcctccttcaaggtgaagagccacgtctacagcctggagggccaggactgcaagtacaccccgatgtttgggcccgaggcccgcaccctggtcctgcgcctcgctcagctcatcacacaggccaaacacacagccaagtccatctccgaccagtgtgcggagagcccggctggccactccttcctctcatggctgggctttagctccatggacaccaatggctcctacacagccaacgacctggacgagatggggcaagacagtgtccggaagacagatgaatacctggagaaggccctggagtacctgcgccagatattccggctcagcgaagcgcagctcaggcagttcacactcgccttgggcaccacccaggatgagaatggaaaaaagcaactccccgactgcatcgtgggtgaggacggactcatccttacgcccctggggcggtaccagatcatcaatgggctgcgaaggtttgaaattgagtaccagggggacccggagctgcagcccatccggagctatgagatcgccagcttggtccgcacactctttaggctgtcgtctgccatcaaccacagatttgcaggacagatggcggctctgtgttcccgggatgacttcctcggcagcttctgtcgctaccacctcacagaacctgggctggccagcaggcacctgctgagccctgtggggcggaggcaggtggccggccacacccgcggccccaggctcagcctgcgcttcctgggcagttaccggacgctggtctcgctgctgctggccttcttcgtggcctctctgttctgcgtcgggcccctcccatgcacgctgctgctcaccctgggctatgtcctctacgcctctgccatgacactgctgaccgagcgggggaagctgcaccagccctgagaattctgcagtcgacggtaccgcgggcccgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatca

SEQ ID NO: 16 - protein, 11 aa, Bos taurus
KGLPYLEQLFR

SEQ ID NO: 17 - protein, 11 aa, Homo sapiens
KALEYLRQIFR

SEQ ID NO: 18 - protein, 178 aa, Homo sapiens
CLQGRVNPVEYSIVMEFLDPGGPMMKLVYKLQAEDYKFDFPVSYLPGPVKASIQECILPDSPLYHNKVQFTPTGGLGLNLALNPFEYYIFFFALSLITQKPLPVSLHVRTSDCAYFILVDRYLSWFLPTEGSVPPPLSSSPGGTSPSPPPRTPAIPFASYGLHHTSLLKRHISHQTSV

SEQ ID NO: 19 - protein, 69 aa, Homo sapiens
YQKMQSPHAKLEVLHYRLSVSSALYSPAQPSLQALHAYQESFTPTEEHVLVVRLLLKHLHAFANSLKPE

SEQ ID NO: 20 - protein, 51 aa, Homo sapiens
KRAAVPRFVQQKLYLFLQHCFGHWPLDASFRAVLEMWLSYLQPWRYAPDKQ

SEQ ID NO: 21 - protein, 79 aa, Homo sapiens
VSEKWAPFVQENLLMYTKLFVGFLNRALRTDLVSPKHALMVFRVAKVFAQPNLAEMIQKGEQLFLEPELVIPHRQHRLF

SEQ ID NO: 22 - protein, 47 aa, Homo sapiens
SFKVKSHVYSLEGQDCKYTPMFGPEARTLVLRLAQLITQAKHTAKSI

SEQ ID NO: 23 - protein, 41 aa, Homo sapiens
TANDLDEMGQDSVRKTDEYLEKALEYLRQIFRLSEAQLRQF

SEQ ID NO: 24 - protein, 132 aa, Homo sapiens
LILTPLGRYQIINGLRRFEIEYQGDPELQPIRSYEIASLVRTLFRLSSAINHRFAGQMAALCSRDDFLGSFCRYHLTEPGLASRHLLSPVGRRQVAGHTRGPRLSLRFLGSYRTLVSLLLAFFVASLFCVGP

SEQ ID NO: 25 - DNA, 25 bp, Artificial
  forward primer for nSMase3
ggaattctgatgacgactttcggcg

SEQ ID NO: 26 - DNA, 25 bp, artificial
  reverse primer nSMase3
gtcgacgtcagggctggtgcagctt

SEQ ID NO: 27 - DNA, 25 bp, artificial
  forward primer nSMase2
gggatccatggttttgtacacgacc

SEQ ID NO: 28 - DNA, 24 bp, artificial
  reverse primer nSMase2
cctcgagctatgcctcctcctccc

SEQ ID NO: 29 - DNA, 8078 bp, Artificial
  AdCGIpnSMase3; AdCGI vector encoding nSMase3
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggataggatcaagcttatcgataccgtcgacctcgagatgacgactttcggcgccgtggcggaatggcggcttccatctctgaggcgagcgacgctatggatcccacagtggtttgctaagaaggccattttcaactctccactggaggctgctatggcgttccctcacctgcagcagcccagctttctactggctagcctgaaagctgactctataaataagccctttgcacagcagtgccaagacttggttaaagtcattgaggactttccagcaaaggagctgcacaccatcttcccatggctggtagaaagcatttttggcagcctagatggtgtcctcgttggctggaacctccgctgcttacaggggcgcgtgaatcctgtggagtacagcatcgtgatggaatttctcgaccctggtggcccaatgatgaagttggtttataagcttcaagctgaagactataagttcgactttcctgtctcctacttgcctggtcctgtgaaggcgtccatccaggagtgcatcctccctgacagtcctctgtaccacaacaaggtccagttcacccctactgggggccttggtctgaacttggccctgaatccgttcgagtattacatattcttctttgccttgagcctcatcactcagaagccacttcctgtgtccctccacgtccgtacttcagactgtgcctatttcatcctggtggacaggtacctgtcatggttcctgcccaccgaaggcagtgtgcccccaccactctcctccagcccaggggggaccagcccctcaccacctcccaggacaccagccataccctttgcttcctatggcctccaccacactagcctcctaaagcgacacatctctcatcagacgtctgtgaatgcagaccccgcctcccacgagatctggaggtcagaaactctgctccaggtttttgttgaaatgtggcttcatcactattccttggagatgtatcaaaaaatgcagtcccctcatgccaagctggaggttctgcactaccgactcagtgtctccagcgccctctacagccccgcccaacccagcctccaggccctccacgcctaccaagagtcgttcacgcctactgaggagcatgtgttggtggtgcgcctgctgctgaagcacctgcacgcctttgccaacagcctgaagccagagcaggcctcaccctccgcccactcccacgccaccagccccctggaggagttcaaacgggctgctgtcccgaggttcgtccagcagaaactctacctcttcttgcagcattgctttggccactggcccctggacgcatcgttcagagctgtcctggagatgtggctgagctacctgcagccgtggcggtacgcgcctgacaagcaggctccgggcagcgactcccagccccggtgtgtgtcggagaaatgggcaccctttgtccaggagaacctgctgatgtacaccaagttgtttgtgggctttctgaaccgcgcgctccgcacagacctggtcagccccaagcacgcgctcatggtgttccgagtggccaaagtctttgcccagcccaacctggctgagatgattcagaaaggtgagcagctattcctggagccagagctggtcatcccccaccgccagcaccgactcttcacggcccccacattcactgggagcttcctgtcaccctggccaccagcggtcactgatgcctccttcaaggtgaagagccacgtctacagcctggagggccaggactgcaagtacaccccgatgtttgggcccgaggcccgcaccctggtcctgcgcctcgctcagctcatcacacaggccaaacacacagccaagtccatctccgaccagtgtgcggagagcccggctggccactccttcctctcatggctgggctttagctccatggacaccaatggctcctacacagccaacgacctggacgagatggggcaagacagtgtccggaagacagatgaatacctggagaaggccctggagtacctgcgccagatattccggctcagcgaagcgcagctcaggcagttcacactcgccttgggcaccacccaggatgagaatggaaaaaagcaactccccgactgcatcgtgggtgaggacggactcatccttacgcccctggggcggtaccagatcatcaatgggctgcgaaggtttgaaattgagtaccagggggacccggagctgcagcccatccggagctatgagatcgccagcttggtccgcacactctttaggctgtcgtctgccatcaaccacagatttgcaggacagatggcggctctgtgttcccgggatgacttcctcggcagcttctgtcgctaccacctcacagaacctgggctggccagcaggcacctgctgagccctgtggggcggaggcaggtggccggccacacccgcggccccaggctcagcctgcgcttcctgggcagttaccggacgctggtctcgctgctgctggccttcttcgtggcctctctgttctgcgtcgggcccctcccatgcacgctgctgctcaccctgggctatgtcctctacgcctctgccatgacactgctgaccgagcgggggaagctgcaccagccctgagaattctgcagtcgacggtaccgcgggcccgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcatcgatccataacttcgtataatgtatgctatacgaagttatccagatctggttctatagtgtcacctaaatcgtatgtgtatgatacataaggttatgtattaattgtagccgcgttctaacgacaatatgtccatagggcccctacgtcacccgccccgttcccacgccccgcgccacgtcacaaactccaccccctcattatcatattggcttcaatccaaaataaggtatattattgatgatggccgcagcggcccctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggggccgctgcggc

SEQ ID NO: 30 - DNA, 60 bp, Artificial
  sense strand of shRNA against nSMase3
gatctcccctgctgatgtacaccaagttcaagagacttggtgtacatcagcaggttttta

SEQ ID NO: 31 - DNA, 60 bp, Artificial
  antisense strand of shRNA against nSMase3
agcttaaaaacctgctgatgtacaccaagtctcttgaacttggtgtacatcagcagggga

SEQ ID NO: 32 - DNA, 2935 bp, Artificial
  pENTRY-shnSMase3; vector encoding shRNA to nSMase3
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctagcattcctgcagttctagaactagtggatcccccgggctgcaggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacagatctcccctgctgatgtacaccaagttcaagagacttggtgtacatcagcaggtttttaagcttatcgataccgtcgacctcgagggggggcccggtaccaatcgatttaaatagctcgctagcacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaataatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 33 - DNA, 6869 bp, Artificial
  pLenti-shnSMase3; lentiviral vector encoding shRNA to nSMase3
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggatctggcctccgcgccgggttttggcgcctcccgcgtaccgagctcggatccactagtccagtgtggtggaattctgcagatatcaacaagtttgtacaaaaaagcaggctagcattcctgcagttctagaactagtggatcccccgggctgcaggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacagatctcccctgctgatgtacaccaagttcaagagacttggtgtacatcagcaggtttttaagcttatcgataccgtcgacctcgagggggggcccggtaccaatcgatttaaatagctcgctagcacccagctttcttgtacaaagtggttgatatccagcacagtggcggccgctcgagtctagagggcccgcggttcgaaggtaagcctatccctaaccctctcctcggtctcgattctacgcgtaccggttagtaatgagtttggaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

(19) United States
(12) Patent Application Publication
(10) Pub. No.: US 2009/0300778 A1
(43) Pub. Date: Dec. 3, 2009

(54) Neutral Sphingomyelinase-E and Its Use

(76) Inventors: Martin Krönke, Koln (DE); Oleg Krut, Koln (DE); Katja Krönke-Wiegmann, Koln (DE); Uta Hoppe, Koln (DE)

Correspondence Address: Ballard Spahr LLP, SUITE 1000, 999 PEACHTREE STREET, ATLANTA, GA, 30309-3915, US

(21) Appl. No.: 12/223,749

(22) PCT Filed: Feb. 8, 2007
(86) PCT No.: PCT/EP2007/051203

§ 371 Date: Dec. 2, 2008

(30) Foreign Application Priority Data
Feb. 8, 2006 (EP) 06101431.2

(51) Int. Cl. A01K 67/00; A61K 39/395; A61K 38/46; C12Q 1/68; C12Q 1/34; C12Q 1/02; C12P 21/06; C12N 9/14; C12N 15/63; C12N 5/10; C12N 5/00; A61K 31/7088; C07K 16/00; C07H 21/04

(52) U.S. Cl. 800/9; 424/130.1; 424/94.6; 435/6; 435/18; 435/29; 435/69.1; 435/195; 435/320.1; 435/325; 514/44 R; 530/387.9; 536/23.2; 536/24.5; 800/8

(57) ABSTRACT

The present invention provides a neutral sphingomyelinase-3 (nSMase3), a nucleic acid encoding said nSMase3, a vector containing said nucleic acid, and cells and non-human organisms transformed or transfected with said nucleic acid sequence or vector. The invention furthermore relates to the use of said nSMase3 as pharmaceutical or diagnostic agent. Test systems for candidate active agents for their therapeutic potential in diseases connected with nSMase3 are also provided.

The present invention provides a neutral sphingomyelinase-3 (nSMase3), a nucleic acid encoding said nSMase3, a vector containing said nucleic acid, and cells and non-human organisms transformed or transfected with said nucleic acid sequence or vector. The invention furthermore relates to the use of said nSMase3 as pharmaceutical or diagnostic agent. Test systems for candidate active agents for their therapeutic potential in diseases connected with nSMase3 are also provided.

### BACKGROUND

Dilated cardiomyopathy (DCM) is a heart-muscle disease characterized by ventricular dilatation and impaired systolic function. The incidence of DCM is 7/100,000per year, and the prevalence is 37/100,000(Codd, M. B. et al., Circulation 80:564-572 (1989); Rakar, S. et al., Eur. Heart J. 18:117-123 (1997)). DCM is a leading cause of heart failure and the most frequent indication for heart transplantation in young patients (Dec, W. G. and Fuster, V., N. Engl. J. Med. 331:1564-1575 (1994)). Heart failure is a progressive clinical syndrome, with a median survival time of 1.7 years in men and 3.2 years in women (Ho, K. K. L. et al., Circulation 88:107-115 (1993)). Most patients die from pump failure or sudden cardiac death. Although familial occurrence of DCM has been reported for the first time by Whitfield (Whitfield, A. G., Q. J. Med. 3:119-134 (1961)), the frequency of familial etiology has been systematically examined only recently (Mestroni, L. and Giacca, M., Curr. Opin. Cardiol. 12:303-309 (1997)). Systematic examination of relatives showed that probably >25% of DCM cases are of familial etiology (Michels, V. V. et al., N. Engl. J. Med. 326:77-82 (1992); Keeling, P. J. et al., Br. Heart J. 73:417-421 (1995); Grünig, E. et al., J. Am. Coll. Cardiol. 31:186-194 (1998)). The mode of inheritance in most families seems to be autosomal dominant (Michels, V. V. et al., N. Engl. J. Med. 326:77-82 (1992)), but autosomal recessive, X-linked, and mitochondrial transmission have also been reported (Mestroni, L. and Giacca, M., Curr. Opin. Cardiol. 12:303-309 (1997)).

To date more than 15 loci for autosomal dominant DCM have been reported: on chromosomes 1p1-q1 (CMD1A), 9q13-q25 (CMD1B), 10q21-q23 (CMD1C), 1q32 (CMD1D), 3p25-p22 (CMD1E), 6q23 (CMD1F), 2q31 (CMD1G) and 2q14-q22 (CMD1H), mutations in the DES gene on 2q35 (CMD 1I), 6q23-q24 (CMD1J), and 6q12-q16 (CMD1K), in the SGCD gene on 5q33 (CMD1L), in the CSRP3 gene on 11p15.1 (CMD1M), in the TCAP gene on 17q12 (CMD1N), and in the ABCC9 gene on 12p12.1 (CMD1O) (Kass, S. et al., Nat. Genet. 7:546-551 (1994); Durand, J. B. et al., Circulation 92:3387-3389 (1995); Krajinovic, M. et al., Am. J. Hum. Genet. 57:846-852 (1995); Bowles, K. R. et al., J. Clin. Invest. 98:1355-1360 (1996); Olson, T. M. and Keating, M. T. J. Clin. Invest. 97:528-532 (1996); Messina, D. N. et al., Am. J. Hum. Genet. 61:909-917 (1997); Siu, B. L. et al., Circulation 99:1022-1026 (1999); Jung, M. et al., Am. J. Hum. Genet. 65:1068-77 (1999), Li, D. et al., Circulation 100:461-464 (1999); Schonberger, J. et al., Nature Genet. 37:418-422 (2005); Sylvius, N. et al., A. J. Hum. Genet. 68 :241-246 (2001); Tsubata, S. et al., J. Clin. Invest. 106:655-662 (2000); Knoll, R. et al., Cell 111:943-955 (2002); Bienengraeber, M. et al., Nature Genet. 36:382-387 (2004)).

Currently, DCM is not curable. Treatment usually includes drug therapy and is aimed at minimizing symptoms and preventing the development of complications and progression of the disease. The drug therapy includes Angiotensin-converting-enzyme inhibitors, beta-blockers, diuretics and anticoagulants. Some patients deteriorate in spite of treatment and may require pacemaker implantation or cardiac transplantation. Thus, there is a need for new medicaments for therapy of DCM.

Sphingomyelinases (SMases, EC3.1.4.12) are sphingomyelin (SM) phosphodiesterases (SMPD) that catalyze hydrolysis of membrane SM to form ceramide and phosphatidylserine (PS). Ceramide has been suggested to play important roles in cell cycle arrest, apoptosis, inflammation, and the eukaryotic stress response (Gulbins, E. and Kolesnick, R., Oncogene 22:7070-7077 (2003); Hannun, Y. A. and Luberto, C., Trends Cell. Biol. 10:73-80 (2000); Kolesnick, R. N. and Krönke, M., Annu. Rev. Physiol. 60:643-665 (1998); Levade, T. and Jaffrezou, J. P., Biochim. Biophys. Acta 1438:1-17 (1999)). Although ceramide can be generated by de novo synthesis through ceramide synthase, it is generated from SM by the action of neutral or acid SMases for the majority of cellular responses.

Different SMases have been described in eukaryotes and prokaryotes and are distinguished by their subcellular localization, pH optima, and requirement for metal ions. Three mammalian SMases have been molecularly cloned. The best characterized of these enzymes is the acid SMase (SMPD1) that is predominantly located in lysosomes but also secreted in response to different ligand-receptor interactions at the cell surface (Schuchman, E. H. et al., J. Biol. Chem. 266:8531-8539 (1991)). SMPD1-deficiency results in lipid storage disorders that manifest in Niemann Pick disease (Ferlinz, K. et al., Biochem. Biophys. Res. Commun. 179:1187-1191 (1991)). The regulated breakdown of SM to ceramide by activation of SMPD1 has been implicated in numerous cell responses from cell survival to apoptosis (Gulbins, E. and Kolesnick, R., Oncogene 22:7070-7077 (2003); Hannun, Y. A. and Luberto, C., Trends Cell. Biol. 10:73-80 (2000); Kolesnick, R. N. and Krönke, M., Annu. Rev. Physiol. 60:643-665 (1998); Levade, T. and Jaffrezou, J. P., Biochim. Biophys. Acta 1438:1-17 (1999)). Two Mg²⁺ dependent neutral SMases, SMPD2 and SMPD3 were recently identified and cloned based on their sequence homology to bacterial nSMases (Tomiuk, S. et al., Proc. Natl. Acad. Sci. USA 95:3638-3643 (1998); Hofmann, K. et al., Proc. Natl. Acad. Sci. USA 97:5895-5900 (2000), WO 99/07855, EP-A-1204757). As the cDNAs encoding said nSMases were available, the biochemical and functional characterization of said nSMases was possible.

The ubiquitously expressed nSMase1 (SMPD2) is Mg²⁺ dependent, localizes to the endoplasmatic reticulum (ER), and is reversibly inhibited by oxidized glutathione and reactive oxygen species. Recently, it has been shown that nSMase1 has phospholipase C activity towards specific lysophospholipids (Sawai, H. et al., J. Biol. Chem. 274:38131-38139 (1999)). Using an antisense strategy, Tonetti et al. showed that nSMase1 may be involved in ceramide-mediated apoptosis triggered by T cell receptor ligation (Tonnetti, L. et al., J. Exp. Med. 189:1581-1589 (1999)). However, gene-targeted mice deficient for nSMase1 did not show any overt functional phenotype and no accumulation or changed metabolism of SM or other lipids (Zumbansen, M. and Stoffel, W., Mol. Cell. Biol. 22:3633-3638 (2002)). Thus, although some insights into the properties of nSMase1 have been provided, its functional significance is still unknown.

NSMase2 (SMPD3) is expressed predominantly in brain and colocalizes with a Golgi marker in neuronal cells (Hofmann, K. et al., Proc. Natl. Acad. Sci. USA 97:5895-5900 (2000)). NSMase2 has a neutral pH optimum, depends on Mg²⁺ ions, and is activated by unsaturated fatty acids and phosphatidylserine. Biochemical characterization of nSMase2 overexpressed in yeast cells lacking inositol phosphosphingolipid phospholipase (Isc1p) revealed that smpd3 is a structural gene for nSMase2 which acts as a bona fide nSMase in cells (Sawai, H. et al., J. Biol. Chem. 275:39793-39798 (2000); Marchesini, N. et al., J. Biol. Chem. 278:13775-13783 (2003)). Stable overexpression of nSMase2 in MCF7 cells resulted in a markedly decreased growth rate at the late exponential phase suggesting that nSMase2 is involved in the regulation of cell growth (Marchesini, N. et al., J. Biol. Chem. 278:13775-13783 (2003)). In mice, deletions in the gene encoding nSMase2 result in developmental and postnatal abnormalities. Stoffel, A. et al., Proc. Natl. Acad. Sci. USA 101:9079-9084 (2004), demonstrated that gene-targeted mice deficient for nSMase2 develop a novel form of dwarfism and delayed puberty as part of a hypothalamus-induced combined pituitary hormone deficiency suggesting that nSMase2 plays a pivotal role in the control of late embryonic and postnatal development. Immature architecture and delayed ossification of long bones was also observed. Strikingly, positional cloning of the recessive mutation fragilitas ossium (fro) in mice identified a deletion in the gene encoding nSMase2 (Smpd3) that led to complete loss of enzymatic activity (Aubin, I. et al., Nat. Genet. 37:803-805 (2005)). The mouse mutation fragilitas ossium leads to a syndrome of severe osteogenesis and dentinogenesis imperfecta with no collagen defect. At birth, homozygous fro/fro mice are smaller than normal with deformities and multiple fractures of ribs and long bones (Aubin, I. et al., Nat. Genet. 37:803-805 (2005)). Thus, Smpd3 null mice partially recapitulate the pathology observed in homozygous fro/fro mice suffering osteogenesis and dentinogenesis imperfecta. Notably, the precise relationship between the impairment of nSMase2, its cascade of intracellular effects and the production of defective extracellular mineralized tissue is not yet fully understood.

In conclusion, the precise function of individual neutral SMases in SM turnover and in the regulation of ceramide-mediated cellular responses is not well understood up to now. Thus, there was a need for further investigations on mammalian nSMases in order to elucidate their physiological functions and to find new levers for diagnosis and therapy of nSMase-related diseases.

On the other hand from Database EMBL XP002393852 (July 2004) a hypothetical human protein was known which was later (in July 2006) designated “NSMASE3”. Further Database EMBL XP002393852 (October 2000) discloses a hypothetical protein, a major part of which overlaps with the protein of XP002393852. In fact said proteins contain sequences which were found by the present invention as belonging to NSMase3.

### SUMMARY OF THE INVENTION

The present invention provides a novel human sphingomyelinase, nSMase3, which was furthermore cloned and characterized. Said nSMase3 is responsive to extracellular stimuli like TNF. Biochemical and functional analysis revealed that nSMase3 shares biochemical properties with nSMase1 and nSMase2, but is functionally distinct with regard to its subcellular localization to the ER. The invention furthermore provides several non-human nSMase3 proteins from eukaryotes. The features distinguishing the human nSMase3 of present invention from other known SMases are as follows:

(a) the amino acid sequence of human nSMase3 (SEQ ID NO:3) shows no homology with other SMase sequences besides nSMase3 sequences from other organisms;
(b) human nSMase3 is highly expressed in heart, skeletal muscle and testis;
(c) overexpression of nSMase3 in cardiomyocytes leads to increased contractility (positive inotropic effect);
(d) the reduced activity of nSMase3 confers protection to host cells against virus-induced cytotoxicity.

In man, the gene coding for nSMase3 is located on chromosome 2, locus q21. This locus is connected with familial dilated cardiomyopathy 1H, a genetic variant of an autosomal-dominant inheritable disease. Thus, nSMase3 may play a causal role in the pathogenesis of said disease. Furthermore, diagnosis of said disease may be possible by detecting mutations in the nSMase3 gene.

nSMase3-deficient cells, especially cardiomyocytes, and corresponding animal models such as nSMase3-deficient (knock-out) mice provide a unique in vitro and in vivo model for cardiovascular diseases, especially for dilated cardiomyopathy 1H. These models can be used for testing candidate agents for therapy of said diseases.

Moreover, nSMase3 in man and animal presents itself as target for medicaments and for somatic gene therapy.

In view of the severe and mostly fatal course of dilated cardiomyopathy 1H, the lack of therapy for said disease and its unclear etiology, the nSMase3 is a key enzyme for development of novel diagnostic and therapeutic methods.

On the other hand, the high expression levels of nSMase3 in testis identify nSMase3 and its mutants as diagnostic targets for fertility disorders in men. In addition, nSMase3 is a novel candidate target for contraception in men.

Furthermore, the downregulation of nSMase3 mRNA by stable shRNA transfection results in a markedly reduced virus-induced cytotoxicity after infection with vesicular stomatitis virus (VSV).

Finally, the present invention provides a cDNA encoding said nSMase3, smpd4.

In particular, the present invention provides
(1) an isolated neutral sphingomyelinase (nSMase3) having the amino acid sequence represented by one of SEQ ID NOs:3 to 9, or a homologue, functional variant, derivative or fragment thereof which possesses the enzymatic and/or immunologic properties of said nSMase3;
(2) an isolated nucleic acid sequence encoding an nSMase3 as defined in (1) above, or a nucleic acid complementary thereto;
(3) a vector comprising the nucleic acid sequence as defined in (2) above;
(4) a cell being transfected or transformed with the vector as defined in (3) above, and/or heterologously comprising the nucleic acid as defined in (2) above, and/or being capable of heterologously expressing an nSMase3 as defined in (1) above;
(5) a tissue or tissue culture comprising cells being transfected or transformed with the vector as defined in (3) above and/or heterologously comprising the nucleic acid as defined in (2) above and/or heterologously expressing the nSMase3 as defined in (1) above;
(6) a non-human transgenic organism comprising cells being transfected or transformed with the vector as defined in (3) above and/or heterologously comprising the nucleic acid as defined in (2) above and/or heterologously expressing the nSMase3 as defined in (1) above;
(7) use of the cell as defined in (4) above, the tissue or tissue culture as defined in (5) above, or the non-human transgenic organism as defined in (6) above as a test cell system, test tissue or test model organism in testing candidate active agents for their therapeutic potential in diseases connected with nSMase3, preferably in heart muscle diseases, male reduced fertility or infertility, viral infections, or for their potential as contraceptive in men or male animals;
(8) a method for testing candidate active agents for their therapeutic potential in diseases connected with nSMase3, preferably in heart muscle diseases, male reduced fertility or infertility, viral infections, or for their potential as contraceptive in men or male animals, comprising the steps
(a) administering the candidate agent to a culture of the cell as defined in (4) above, to a tissue or tissue culture as defined in (5) above, or to the non-human organism as defined in (6) above; and
(b) determining the effect of the candidate agent on said cell, tissue or non-human organism;
(9) a method for preparing the nSMase3 as defined in (1) above, which method comprises culturing the cells as defined in (4) above and isolating the nSMase3 from the culture;
(10) an antibody specific for the nSMase3 as defined in (1) above;
(11) a shRNA or siRNA against nSMase3;
(12) a vector encoding the shRNA or siRNA of (11) above;
(13) a pharmaceutical or diagnostic composition which comprises one or more of the following: the isolated nSMase3 of (1) above, the nucleic acid of (2) above, the vector of (3) and (12) above, the transformed or transfected cell of (4) above, the antibody of (10) above, and the shRNA or siRNA of (11) above, and pharmaceutically or diagnostically acceptable salts and derivatives thereof;
(14) use of the isolated nSMase3 of (1) above, the nucleic acid of (2) above, the vector of (3) above, and/or pharmaceutically acceptable salts or derivatives thereof for preparing a medicament for prevention or treatment of heart muscle diseases, male reduced fertility or infertility;
(15) use of the antibody of (10) above, the shRNA and siRNA of (11) above, the vector of (12) above, other nSMase3 inhibitors including other RNAi agents, and/or pharmaceutically acceptable salts or derivatives thereof for preparing a medicament for (a) prevention or treatment of diseases connected with increased activity of nSMase3, preferably cramps, tremor, Parkinson's disease and hypertrophic cardiomyopathy, or (b) contraception in men or male animals, or c) viral infections;
(16) a method for diagnosing and/or monitoring a disorder characterized by changes in the expression of an nSMase3 as defined in (1) above, which method comprises the steps
(a) isolating a biological sample from a subject having or suspected to have said disorder;
(b) detection of the amount and/or activity of said nSMase3 or of the nucleic acid sequence encoding said nSMase3, and/or of mutations present in said nucleic acid sequence in comparison to the native nSMase3 encoding sequence;
(17) a kit for performing the diagnostic method of (16) above, comprising one or more of the following: the nSMase3 of (1) above, the nucleic acid of (2) above, the vector of (3) above, the cell of (4) above, the antibody of (10) above and diagnostically acceptable salts and derivatives thereof;
(18) an isolated cell, tissue or tissue culture, or a non-human transgenic organism, having a reduced abundance of nSMase3 or being deficient in nSMase3 or in the nSMase3 gene, or comprising a siRNA or shRNA of (11) or a vector of (12) above;
(19) the use of the cell, tissue or tissue culture, or the non-human organism of (18) above
(i) as a model for a disease connected with a deficiency of nSMase3; or
(ii) for the generation of antibodies to nSMase3, preferably for the generation of monoclonal antibodies including the antibodies of (10) above, and/or of antibodies capable of modulating nSMase3 activity;
(20) the use of agents reducing the abundance of nSMase3 in an organism for preparing a medicament for the treatment of diseases connected with increased heart or skeletal muscle contractility, viral infections and for reducing the fertility in male patients or male animals;
(21) a method for preventing or treating heart muscle diseases, especially DCM, or male reduced fertility or infertility, or viral infection, which method comprises administering to the patient an effective dose of one or more of the following: the nSMase3 of (1) above, the nucleic acid of (2) above, the vector of (3) above, and pharmaceutically acceptable salts and derivatives thereof;
(22) a method (a) for preventing or treating heart muscle diseases connected with increased activity of nSMase3, preferably hypertrophic cardiomyopathy, or (b) for reducing the fertility in a male patient or male animal, or c) for preventing or treating viral infections, which method comprises administering to the patient an effective dose of one or more of the following: an nSMase3 inhibitory agent, the antibody of (10) above, the shRNA and siRNA of (11) above, the vector of (12) above, any other RNAi agent to nSMase3, and pharmaceutically acceptable salts and derivatives thereof; and
(23) a method for downregulating the expression of nSMase3 in vivo and in vitro by RNA interference (RNAi), preferably by administering to the target cell or organism one or more of the group consisting of the siRNA or shRNA of (11) above or the vector of (12) above.

### BRIEF DESCRIPTION OF THE FIGURES

FIG. 1: Comparison of the sequence of nSMase3 with functional analogs and homologous proteins.

A) Deduced amino acid sequence of human nSMase3 aligned with nSMase1 and nSMase2 (Genbank access numbers NP_{—}003071 and NP_{—}061137, respectively). Positions of amino acids are numbered on the left. Grey shaded boxes indicate identical or similar amino acids, highly conserved amino acids are shaded in black. The solid frame indicates the putative transmembrane domain, the dashed frame indicates the match with bovine peptide sequences.

B) Proteins with significant amino acid sequence homology identified by BLAST search of the Genbank database. When more then one sequence from the same species were identified, sequences with highest degree of similarity were preferred. The sequence of human nSMase3 (SEQ ID NO:3) was aligned with deduced amino acid sequences of homologous proteins from dog (XP_{—}543560; SEQ ID NO:4), mouse (BAB31737; SEQ ID NO:5), chicken (XP_{—}415; SEQ ID NO:6), zebrafish (XP_{—}706729; SEQ ID NO:7), drosophila (NP_{—}650124; SEQ ID NO:8) and C. elegans (NP_{—}492324; SEQ ID NO:9) using ClustalW algorithm.

FIG. 2: Expression of nSMase3 mRNA in human tissues. Poly(A)+ mRNA from different human tissues (Clontech human 12 lane MTN (A) and human MTN II blot (B)) were analyzed by Northern blotting using as a hybridization probe a random-primed ³²P—labelled full-length nSMase3 cDNA.

FIG. 3: Overexpression of nSMase3 in 293 cells and MCF7 cells as described in example 5. A,B) 293 and MCF7 cells were transiently transfected with increasing doses of an expression plasmid, pRK-nSMase3 (SEQ ID NO:10) encoding full-length human nSMase3. 36 h after transfection, membrane fractions were assayed for nSMase activity. C) SM hydrolysis in nSMase3-transfected cells. MCF7 cells were transiently transfected with pRK-nSMase3 and pRK-nSMase2 expression vectors (SEQ ID NOs:10 and 11). Membrane fractions were analyzed for SM levels. D) Ceramide generation by nSMase3-transfected cells. Organic fractions of MCF7 cells left untreated or transfected with pRK-nSMase3 were prepared and ceramide generation was determined by DAG-kinase followed by TLC analysis.

FIG. 4: Overexpression of nSMase3 in S. cerevisiae. NSMase3 as well as nSMase2 (control), respectively, were subcloned in the YES2 expression vector, and the resulting pYES-nSMase-vector (pYES-nSMase3, SEQ ID NO:12; pYES-nSMase2, SEQ ID NO: 13) was transiently transfected into JK9-3dΔIsc1 yeast cells. A) SM hydrolysis was measured in microsomal fractions as described in example 6A and in the presence of 6.7 mol % Phosphatidylserine (PS) (100 μM). B) SM hydrolysis was measured for microsomal fractions of nSMase3 transfected yeast cells at increasing concentrations of SM and in the presence of 6.7 mol % PS (100 μM). Shown are results from one experiment which is representative for two separate experiments.

FIG. 5: Characterization of nSMase3 in yeast. nSMase activity was measured in microsomal fractions of JK9-3dΔIsc1p cells transiently transfected with pYES-nSMase3 (SEQ ID NO:12) in the presence of graded concentrations of the indicated cations (A) or PS (B). C) Microsomal fractions from nSMase3 transfected yeast cells were preincubated for 30 min at 37° C. with indicated concentrations of scyphostatin and assayed for nSMase activity. The values shown represent the average of duplicates. Similar results were obtained in two separate experiments.

FIG. 6: TNF responsiveness of nSMase3. A) To obtain stable nSMase3 overexpressors, MCF7 cells were transfected with pRK-nSMase3 (SEQ ID NO:10) and with BMGneo coding for neomycin resistance (Karasuyama, H. and Melchers, F., Eur. J. Immunol. 18:97-104 (1988)). NSMase3 overexpressors were selected by culture in the presence of 800 μg/ml G418 and cloning by limiting dilution. B) Parental MCF7 cells and MCF7 cells stably transfected with nSMase3 (clone23), were stimulated in duplicates with TNF-α (100 ng/ml). At the indicated periods of time membrane fractions were analyzed for nSMase activity. The results are representative for three independent experiments.

FIG. 7: Subcellular localization of nSMase3. A) HeLa cells were transiently transfected with an expression vector, pGFP-nSMase3 (SEQ ID NO:15), that encodes Green Fluorescent Protein (GFP) fused to the N-terminus of nSMase3. After 36 h, cells were stained with rat anti-KDEL antibody and Cy3-conjugated goat anti-rat Ig was used as secondary reagent. Cells were counterstained with Hoechst 33258. B) enzymatic activity of nSMase3-GFP fusion protein (GFP at the N-terminus of nSMase3) in 293 cells (see example 10).

FIG. 8: Enzymatic activity in nSMase3 overexpressing MCF7-nSMase3 clone 23 (black bars) or parental MCF7 (grey bars) cells at pH values ranged from 3.0 to 11.0 (example 13). Protein extracts prepared from MCF7-nSMase3 clone 23 (example 5) and parental MCF7 cells (grey bars) were submitted to a sphingomyelinase activity assay as described in example 6A.

FIG. 9: Effect of overexpression of nSMase3 on the contractility of cardiomyocytes. Cardiomyocytes were transfected with nSMase3 cDNA using viral vectors as described in example 11. Contractility was measured as described in example 11. Systolic shortening of nSMase3-overexpressing (AdCGIpnSMase3), of mock transfected (AdCGI), and of nontransfected cardiomyocytes is shown.

FIG. 10: Inhibition of nSMase3 activity by shRNA expression. Neutral sphingomyelinase activity was measured in HeLa cells, which were transfected with shRNA against BAX, DDIF or nSMase3 as described in example 12. Empty vector transfected cells served as a control. Four different clones of shRNA-nSMase3 transfected HeLa cells (clone Nos. 1-4) were investigated. Mean and standard deviation of the activity from 3 independent experiments are shown.

FIG. 11: Hela cells expressing nSMase3 siRNA are less susceptible to VSV infection. Survival of wt HeLa cells, HeLa cells, which were transfected with shRNA against nSMase3, or HeLa cells transfected with shRNA against DDIF protein as described in example 12. 3×10³ Hela cells were incubated overnight with Vesicular Stomatitis Virus (VSV). HeLa cell viability was assessed by the XTT assay (Roche). Values were normalized to non-infected controls and presented as % survival. Mean and standard deviation from 6 independent experiments are shown.

Sequence Listing—Free Text

| SEQ ID NO: | Sequence description |
|---|---|
| 1 | nSMase3 cDNA; human |
| 2 | nSMase3 coding sequence; human |
| 3 | nSMase3 protein; human |
| 4 | nSMase3 protein; dog |
| 5 | nSMase3 protein; mouse |
| 6 | nSMase3 protein; chicken |
| 7 | nSMase3 protein; zebrafish |
| 8 | nSMase3 protein; drosophila |
| 9 | nSMase3 protein; C. elegans |
| 10 | pRK-nSMase3 |
| 11 | pRK-nSMase2 |
| 12 | pYES-nSMase3 |
| 13 | pYES-nSMase2 |
| 14 | AdCGI |
| 15 | pGFP-nSMase3 |
| 16 | bovine oligopeptide nSMase3_bovine_0 |
| 17 | human oligopeptide nSMase3_human_0 |
| 18 | human peptide nSMase3_human_1 |
| 19 | human peptide nSMase3_human_2 |
| 20 | human peptide nSMase3_human_3 |
| 21 | human peptide nSMase3_human_4 |
| 22 | human peptide nSMase3_human_5 |
| 23 | human peptide nSMase3_human_6 |
| 24 | human peptide nSMase3_human_7 |
| 25 | forward primer nSMase3 |
| 26 | reverse primer nSMase3 |
| 27 | forward primer nSMase2 |
| 28 | reverse primer nSMase2 |
| 29 | AdCGIpnSMase3 |
| 30 | sense shRNA against nSMase3 |
| 31 | antisense shRNA against nSMase3 |
| 32 | pENTRY-shnSMase3 |
| 33 | pLenti-shnSMase3 |

### DEFINITIONS

In the framework of the present invention the following abbreviations, terms and definitions are used.

The abbreviations used are: ER, endoplasmatic reticulum; GFP, green fluorescent protein; PC, phosphatidylcholine; PE, phosphatidylethanolamine; PS, phosphatidyl-serine; SM, sphingomyelin; SMase, sphingomyelinase; nSMase, neutral sphingo-myelinase; SMPD, sphingomyelin phosphodiesterase; TNF, tumor necrosis factor.

In the context of present invention, the term “nSMase3” includes the native nSMase3, but also homologues, functional variants, derivatives and fragments thereof, provided that said homologues, functional variants, derivatives and fragments possess the enzymatic and/or immunologic properties of the native nSMase3. In a preferred embodiment of the invention, “nSMase3” refers to a native nSMase3 and its amino acid sequence, more preferably to the nSMases of the group consisting of SEQ ID NOs:3 to 9, most preferably to the nSMase3 of SEQ ID NO:3.

A “homologue” in the context of present invention designates a protein or peptide which possesses the enzymatic and/or immunologic properties of the native nSMase3, but differs from said native nSMase3 in its amino acid sequence in that one or more amino acids are substituted in comparison to the native nSMase3 sequence. Preferably, the said amino acid substitutions are conservative, i.e. the function and/or conformation of the protein is not affected by the substitutions. More preferably, one or more of the amino acids of the native protein are substituted by amino acids with similar chemical properties, e.g. Val by Ala (conservative amino acid exchange). The ratio of substituted amino acids in comparison to the native sequence is preferably from 0 to 30% of the total amino acids in the sequence, more preferably from 0 to 15%, most preferably from 0 to 5%.

A “derivative” of the nSMase3 of the invention encompasses proteins derived from the native nSMase3 amino acid sequence by substitution, addition and/or deletion of one or more amino acids. Said substitutions are preferably conservative as defined above. Said addition products have amino acid sequences resulting from addition of 1-100, preferably 1-40, more preferably of 1-20 amino acids to the native nSMase3 sequence. The added amino acids may be added as single amino acids or in continuous segments containing 2 or more linked amino acids. Said addition may take place at the N- and/or C-terminus and/or inside of the native nSMase3 sequence. More than one addition per sequence are allowed, but a single addition is preferred. Moreover preferred is an addition at the N- or C-terminus, and of these an addition at the N-terminus. The deletion products of the full length native nSMase3 amino acid sequences result from deletion of 1-100, preferably 1-50, more preferably 1-20 amino acids. The deleted amino acids may be deleted as single amino acids or in continuous segments containing 2 or more linked amino acids. Said deletion may take place at the N- and/or C-terminus and/or inside of the native nSMase3 sequence. More than one deletion per sequence are allowed, but a single deletion is preferred. Moreover preferred is a deletion at the N- or C-terminus, and of these a deletion at the N-terminus.

An nSMase3 “fragment” is derived from the native nSMase3 amino acid sequence by deletion of one or more amino acids at the N- and/or C-terminus. Said fragment must still possesses the enzymatic and/or immunologic properties of the native nSMase3. Thus, the minimum length of said fragment is determined by its ability to be recognized and bound by an antibody which is specific for the native protein. Preferably, said fragment has up to 100, more preferably up to 50, most preferably up to 20 amino acids and contains at least 7 amino acids, more preferably at least 15 amino acids.

A “functional variant” of a protein or peptide of the invention is a fusion protein of said protein or peptide (“nSMase3 domain”) with at least one second functional protein domain or functional non-proteinaceous component (“second domain”). Said non-proteinaceous second domain may be selected from non-native chemical components (such as polyethers including PEG, polyesters, alkylation products, etc.), polysaccharides and lipids. Said second protein domain is preferably a marker protein, an immunological adjuvant, a protein conferring antibiotic resistance to a transformed cell, or a ligand for a cell surface receptor expressed by the target cell.

A “nucleic acid sequence encoding a nSMase3” may be any nucleic acid, preferably DNA or RNA, coding for the proteins or peptides of present invention, i.e. for anyone of the group consisting of the nSMase3 of SEQ ID NOs:3 to 9, their functional variants, derivatives, homologues and fragments. Said nucleic acid sequence is either identical or substantially identical to the native sequence of nSMase3 or to the nucleic acid sequence corresponding to the amino acid sequence of the functional variants, homologues, derivatives and fragments of present invention. In the context of present invention, a specific nucleic acid sequence refers to said sequence itself and nucleic acid sequences which are substantially identical thereto. A “substantially identical” nucleic acid sequence differs from the initial nucleic acid only by degenerated codon exchange. Thus, in a substantially identical nucleic acid sequence the codons inside the coding sequences are changed in a way which will not lead to a change of the amino acid sequence of the translation product (i.e. replacement of a codon by another codon out of its codon family). The nucleic acid sequences coding for native nSMase3, more preferably the sequence of SEQ ID NOs:1 and 2, and for nSMase3 derivatives and fragments are preferred in the context of present invention. More preferred are nucleic acid sequences encoding native nSMase3s, especially the nSMase3s of SEQ ID NO:3 to 9.

Preferably, said nucleic acid sequence comprises the cDNA sequence of SEQ ID NO:1, i.e. the cDNA of native human nSMase3, or is derived therefrom by degenerated codon usage. Alternatively preferred is a nucleic acid sequence comprising SEQ ID NO:2 or its exon parts, i.e. the native coding sequence for human nSMase3.

The nucleic acid sequences of the invention are preferably used as full length sequences. However, they may also be used as addition or deletion mutants of said full length sequences for the performance of present invention. The allowable additions and deletions in the nucleic acids of the invention correspond in their range and nature to the allowable amino acid additions and deletions as outlined above. In addition to the deletion or addition of complete codons the addition and deletion of single or pairwise nucleotides is possible.

A “nucleic acid sequence fragment” is a part of the full length nucleic acid sequence which is shorter that full length, but which is still able to hybridize under stringent conditions to the full length nucleic acid sequence. It contains preferably at least 15 nucleotides, more preferably at least 25 nucleotides.

“Native” is used synonymously to “naturally occurring”.

The term “isolated” means that a molecule selected from the group comprising proteins, peptides and nucleic acids is/has been separated or purified from other molecules of said group, wherein said other molecules are usually associated with said isolated molecule in its organism of origin. This encompasses biochemically purified molecules, recombinantly produced and chemically synthesized molecules of said group. An “isolated cell” is a cell which is separate from its organism of origin. An “isolated tissue” is a tissue which is separate from its organism of origin, preferably a primary tissue.

The term “animal” encloses all animals with the exception of humans. Preferred animals in the context of present invention are vertebrates.

The term “vertebrate” according to the present invention relates to an animal having a backbone or spinal column including mammals, birds, reptiles, amphibians and fish. Preferred vertebrates are mammals including humans, birds and fish. Inasfar as transgenic animals are concerned, rodents, especially mice or rats, are preferred for performance of present invention.

“Tissue culture” according to the present invention refers to animal tissue, organ primordia, or the whole or part of an organ which is maintained or grown in vitro so as to preserve its architecture and/or function. Preferred tissue cultures in present invention are from vertebrate tissue. Furthermore, said tissue culture is preferably a primary tissue culture.

A “cell culture” according to the present invention includes isolated cells which are cultured in vitro. These cells can be immortalized by fusion with tumor cells (tumor derived cells) or obtained directly from their native source (primary cell culture). They may be transformed or untransformed.

The term “pharmaceutically acceptable salt(s)”, as used herein, means those salts of compounds of present invention which are safe and effective for topical or systemic use in mammals and that possess the desired biological activity. The counter ion is suitable for the intended use, non-toxic, and it does not interfere with the desired biological action of the pharmaceutical composition. Pharmaceutically acceptable salts in the context of present invention include the salts reviewed in the IUPAC Handbook of Pharmaceutically Acceptable Salts (Wermuth, C. G. and Stahl, P. H., Pharmaceutical Salts: Properties, Selection and Use—A Handbook, Verlag Helvetica Chimica Acta (2002)). Said salts include also the corresponding solvates.

Accordingly, the term “diagnostically acceptable salt(s)” refers to those salts of compounds of present invention which are useful and effective for the desired diagnostic method. Their counter ion does not interfere with the reaction necessary for detection of the target compound. Said salts include also the corresponding solvates. “Pharmaceutically or diagnostically acceptable derivatives” of the compounds of present invention are derivatives formed from the compounds by chemical modification. Said derivatives do not interfere with the pharmaceutical or diagnostic activity of said compounds. In case of proteins and peptides, they include functional variants of proteins as defined above. In case of nucleic acid molecules, they include nucleic acid molecules derivatized with marker molecules (like biotin or radioactive isotopes), connected to further nucleic acids (like, e.g., His-tags) etc.

### DETAILED DESCRIPTION OF THE INVENTION

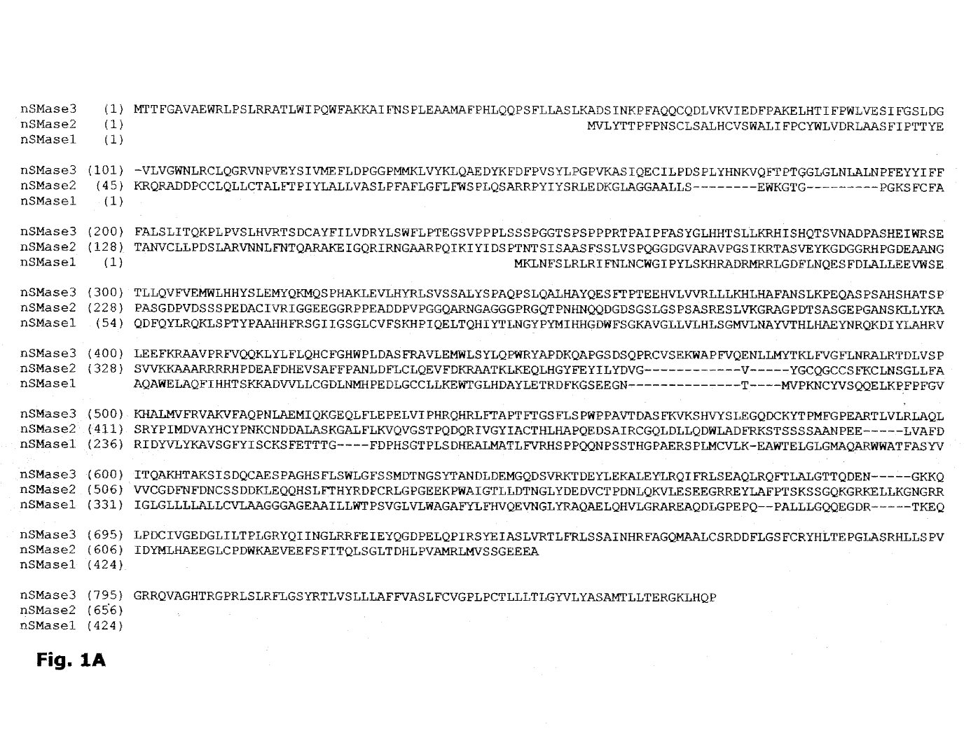

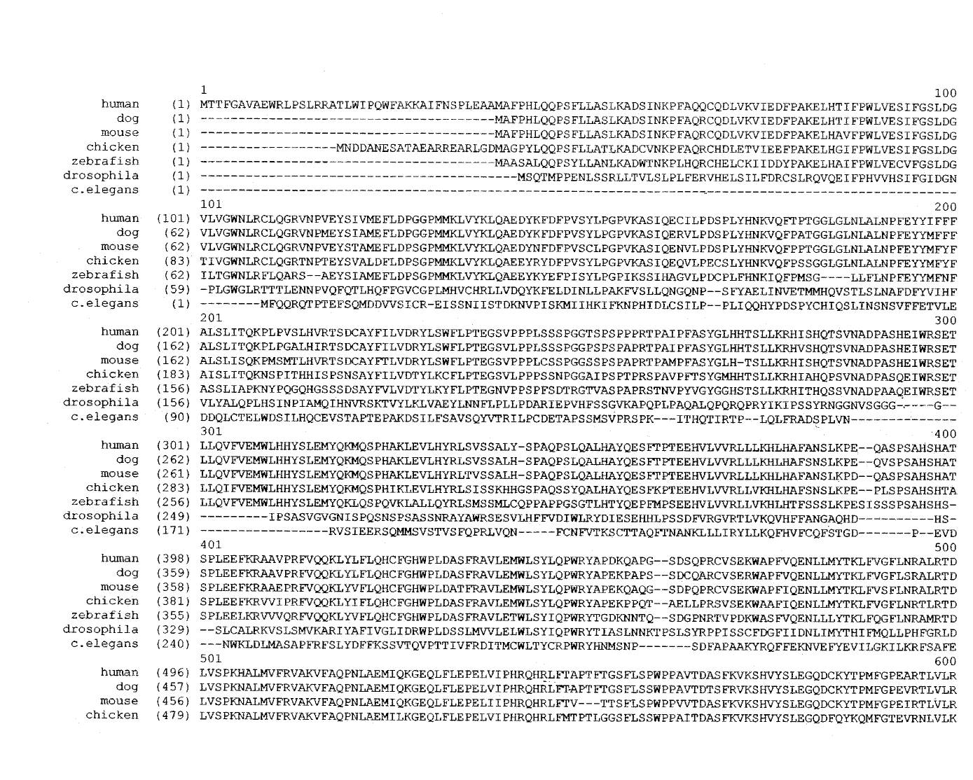

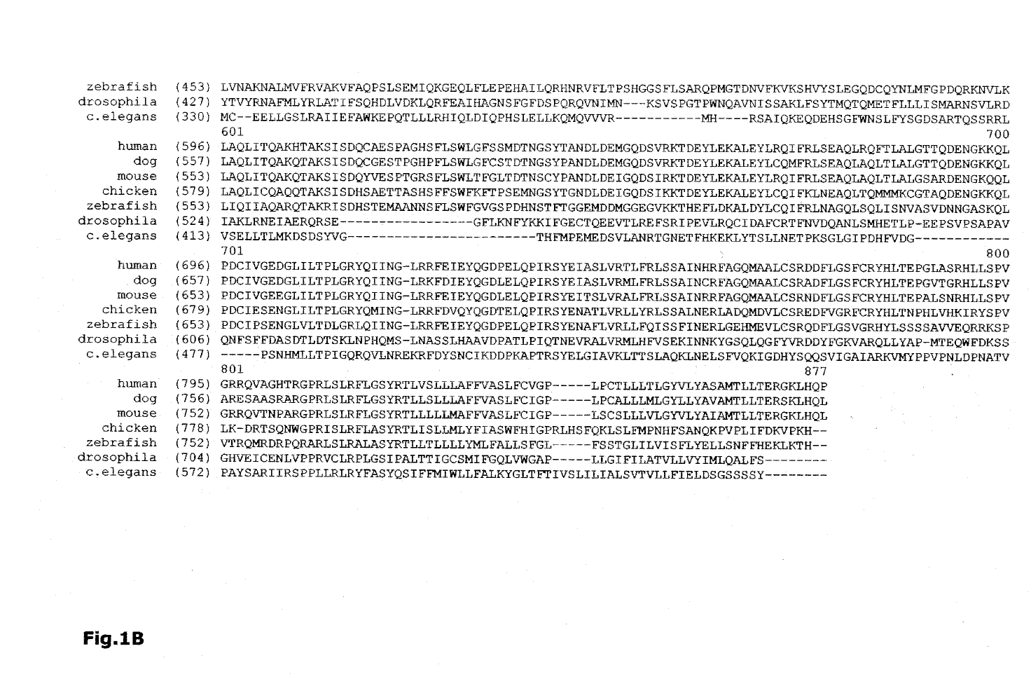

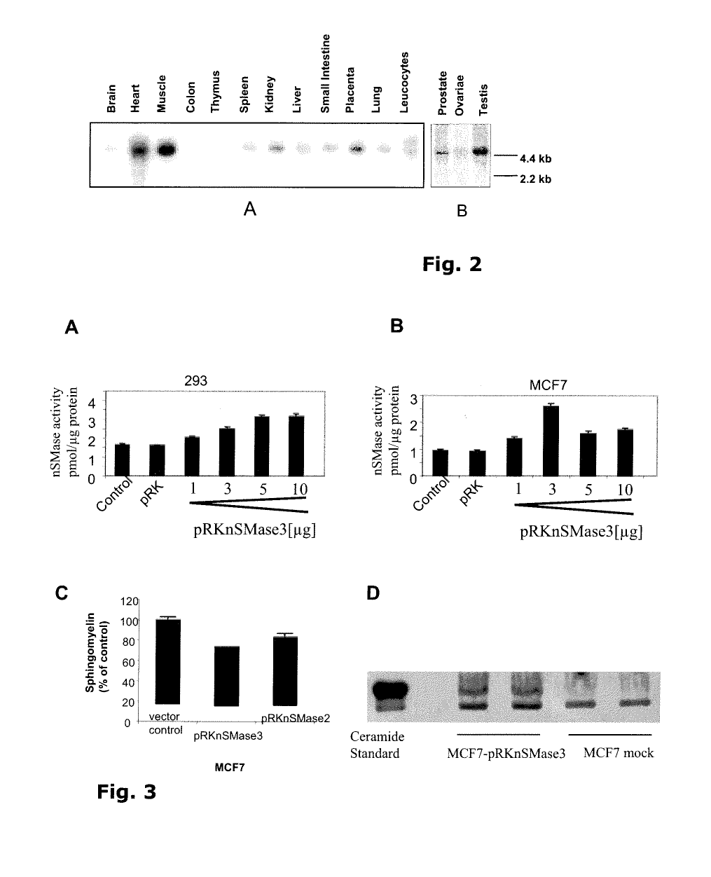

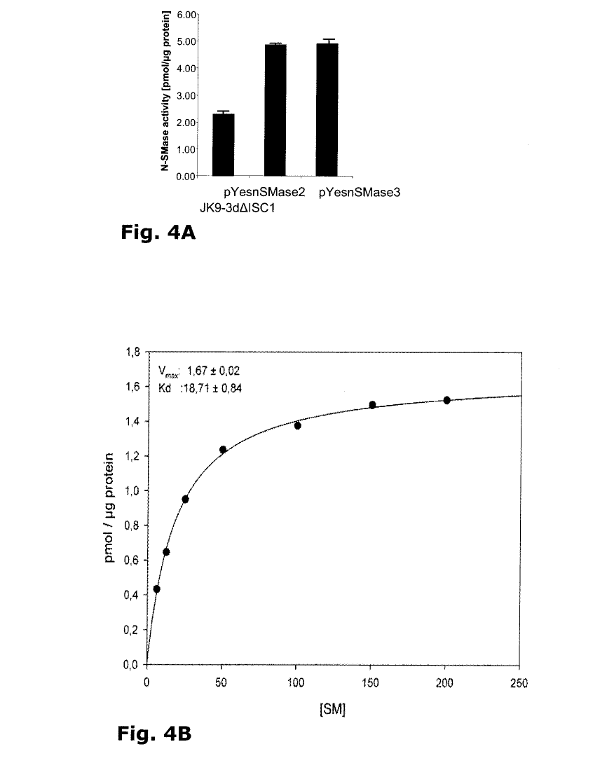

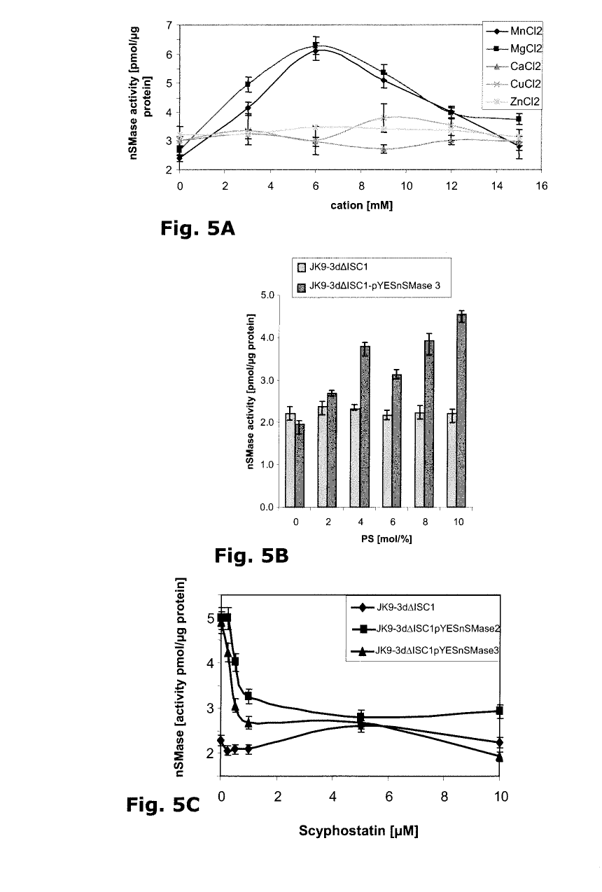

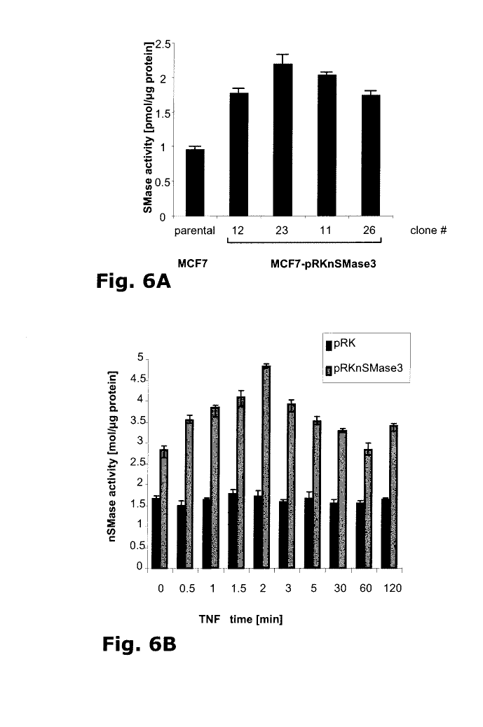

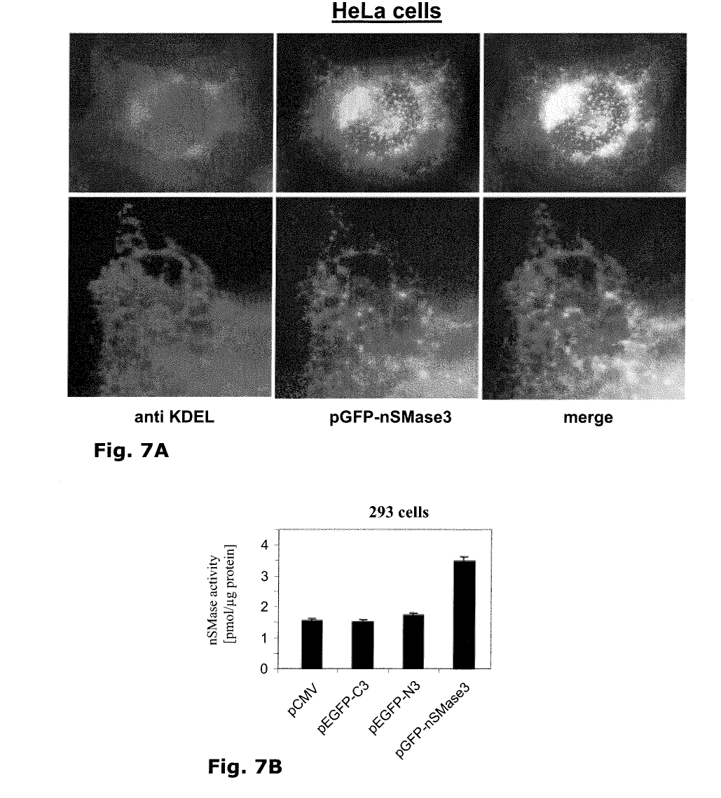

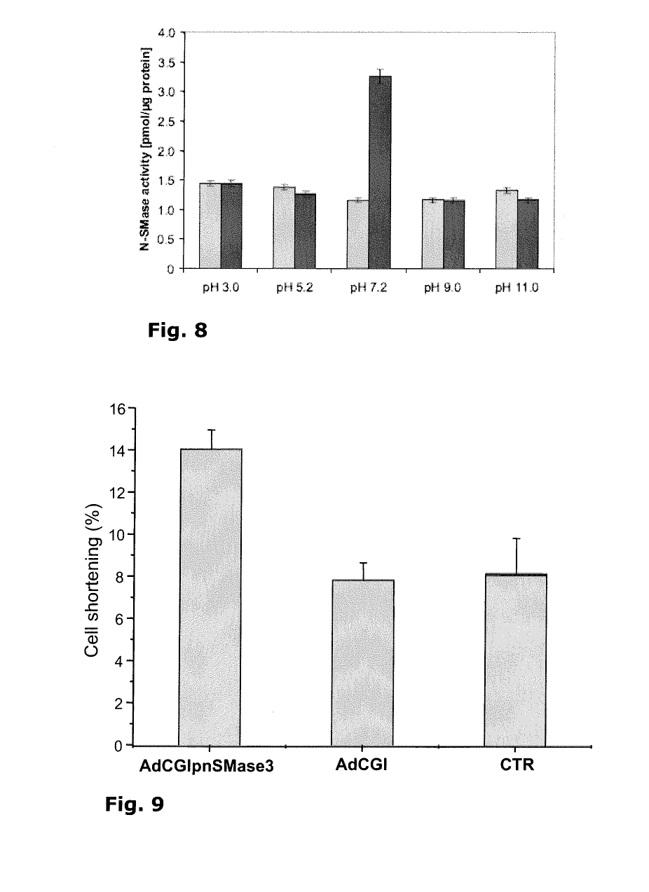

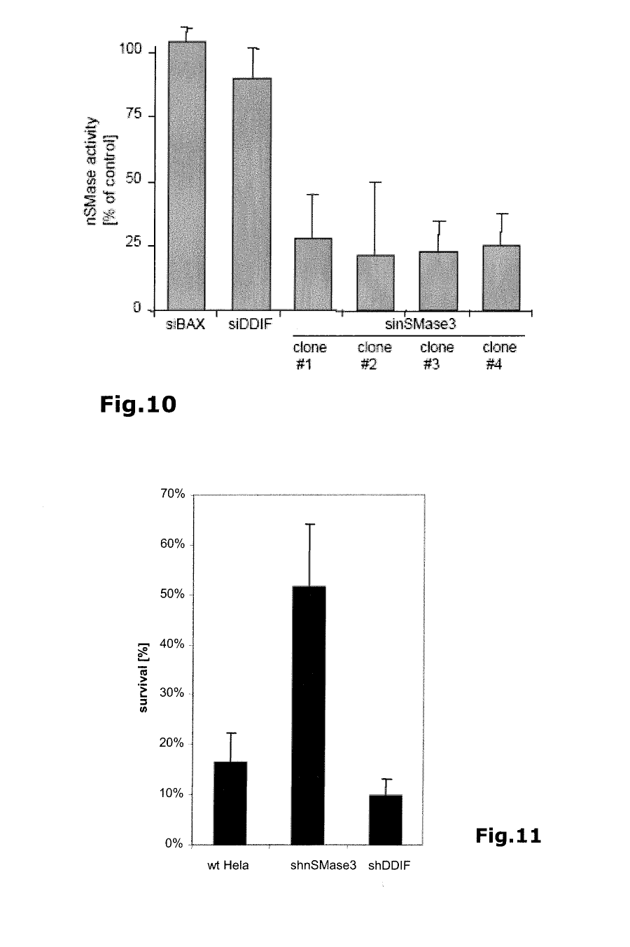

The present invention relates to nSMase3 and its use. Generally, the nSMase3 of embodiment (1) of present invention shows only little sequence homology to nSMase1 and nSMase2 (FIG. 1A). In one preferred aspect of embodiment (1) said nSMase3 consists of the amino acid sequence represented by one of SEQ ID NOs:3 to 9, or is a homologue thereof which possesses the enzymatic and/or immunologic properties of said nSMase3. In a further preferred aspect of embodiment (1) said nSMase3, its homologue, functional variant, derivative or fragment thereof contains an amino acid sequence as represented by SEQ ID NO:16 or 17 (nSMase3_bovine_{—}0, nSMase3_human_{—}0), preferably contains an amino acid sequence as represented by SEQ ID NO:23 (nSMase3_human_{—}6; contains amino acid sequence of SEQ ID NO:17); more preferably contains one or more of the amino acid sequences as represented by SEQ ID NOs:18 to 24 (nSMase3_human_{—}1 to nSMase3_human_{—}7). In another preferred aspect of embodiment (1), said nSMase3, its homologue, functional variant, derivative or fragment thereof lacks an N-terminal leader signal sequence.

In another preferred aspect, the nSMase3 according to embodiment (1) is a vertebrate nSMase3, preferably a mammalian, avian or fish nSMase3, more preferably an nSMase3 as represented by one of SEQ ID Nos:3 to 7. Even more preferably, the nSMase3 is mammalian and/or selected from the group comprising SEQ ID Nos:3 to 5. Most preferably, the nSMase3 according to embodiment (1) is human nSMase3, a homologue, functional variant, derivative or fragment thereof. Especially preferred is the human nSMase3 represented by SEQ ID NO:3 or a homologue thereof.

In a further preferred aspect of embodiment (1) said nSMase3, its homologue, functional variant, derivative or fragment thereof preferably
contains a transmembrane domain close to the C terminus, and/or
is Mg²⁺ dependent, and/or
is activated by TNF, and/or
has a pH optimum at neutral pH, and/or
colocalizes with ER markers.

The pH optimum preferably lies at a value from pH 6.8 to 7.6, more preferably at pH 7.2.

The isolated nucleic acid of embodiment (2) of the invention is a nucleic acid sequence encoding an nSMase3 as defined above in the “definitions” section. Thus, it has the preferred aspects as listed in this section. It is preferably a DNA or RNA. More preferably, it encodes a native nSMase3. Most preferably, it comprises the sequence of SEQ ID NO:1 or 2, its RNA analogon or a sequence complementary thereto.

The vector of embodiment (3) may be any state of the art vector which is suitable for transformation or transfection of procaryotic or eucaryotic cells. It is preferably an expression vector, more preferably a vector which allows regulated, i.e. inducible expression. Most preferred are the vectors used in the examples section.

The transfected or transformed cell of embodiment (4) is preferably an isolated cell. In a preferred aspect of embodiment (4), said transfected or transformed cell is selected from the group consisting of (a) mammalian cells, preferably human or murine cells, more preferably is a cardiomyocyte, myocyte, neural cell, macrophage, granulocyte, spermatogonia, Sertoli cell, Leydig cell or a tumor-transformed cell line thereof, (b) microbial cells, preferably bacterial or yeast cells, (c) insect cells and (d) plant cells. Most preferably, said cell is a primary cell cultured from tumor transformed (i.e. immortalized) cells, more preferably is derived from HeLa, HEK293, MCF7, cardiomyocytes, myocytes, neuronal cells, macrophages, granulocytes, spermatogonia, Sertoli cell or Leydig cell.

In a further preferred aspect of embodiment (4), said cell is overexpressing nSMase3. The non-human organism according to embodiment (6) of the invention is preferably a vertebrate, an invertebrate or a plant. Said organism is more preferably a vertebrate, especially a non-human mammal, more preferably is a rodent, most preferably is a mouse or rat.

The tissue or tissue culture of embodiment (5) of the invention is preferably derived from the non-human organism as defined above. Tissue or tissue culture of human origin is also contemplated by the present invention. Furthermore, the tissue or tissue culture is preferably primary tissue or tissue culture, or it is a tissue culture of tumor transformed cells. Said tumor transformed cells are preferably derived from HeLa, HEK293, MCF7, cardiomyocytes, myocytes, neuronal cells, macrophages, granulocytes, spermatogonia, Sertoli cells or Leydig cells. In a further preferred aspect of embodiment (5), the cells contained in the tissue or tissue culture are overexpressing nSMase3.

The preferred diseases connected with nSMase3 in the use of embodiment (7) and the method of embodiment (8) are heart muscle diseases, viral infections, and male reduced fertility or infertility. This is due to the fact that nSMase3 in humans is mainly expressed in heart and testis. Furthermore, the results of the nSMase3 overexpression in cardiomyocytes indicate a positive inotropic effect of nSMase3. Moreover, it was shown that the downregulation of nSMase3 expression reduces host cellcytotoxicity after infection with certain viruses such as VSV, which is most probably due to reduced uptake of viral particles. This in turn could be explained that viruses use nSMase3 directly or indirectly for the entry host cell as has been shown for the acid sphingomyelinase (Grassme H. et al., J Biol Chem 2005, 280:26256-62). VSV enters the host cell by the clathrin-mediated endocytosis, the most common used pathway of viral infection (Marsh et al., Cell 2006; 124: 729-740).

The use (7) and method (8) are moreover suitable for the search for contraceptives in men or male animals, as testis is one of the few expression sites of nSMase3 in humans and animals.

In the method of embodiment (8), the candidate active agent is administered to the cell, tissue or non-human organism in a suitable preparation or formulation. The dosages which may be administered in the test to a non-human organism are dependent on the specific animal, its size, age and health condition. Thus, they have to be determined individually from case to case. Furthermore, the determination of a dose-effect ratio by testing different dosages is advisable.

The method of embodiment (8) is apt for identification of either suppressors or enhancers of nSMase3, and even of substituents for nSMase3. This is due to the fact that it may be performed not only with the cells/tissues/animals of embodiments (4) to (6), but also with cells/tissue/animal lacking a part or all of their native nSMase3, especially the cells/tissue/animal of embodiment (18). In the latter case, said cells/tissue/animal serve as a model for a disease connected with an nSMase3 deficiency.

The effect determination of embodiment (8)(b) is preferably done or supplemented by detection of the amount and/or activity of nSMase3 or of the nucleic acid sequence encoding nSMase3 as described for embodiment (16)(b) below.

The method (9) is preferably performed with yeast or mammalian cells, more preferably with S. cerevisiae cells or immortalized mammalian cells, most preferably with immortalized human cells including HEK293, MCF7, HeLa and immortalized cardiomyocytes, myocytes, neuronal cells, macrophages, granulocytes, spermatogonia, Sertoli cells or Leydig cells. The isolation of the nSMase3 produced by said cells may be performed by any textbook method for protein isolation and purification, but is preferably done by chromatography (including ion-exchange chromatography, hydrophobic interaction chromatography, affinity chromatography). The antibody of embodiment (10) is preferably an antibody raised against the nSMase3 of embodiment (1). It is preferably raised in non-human animals lacking nSMase3 and/or using nSMase3 epitopes which are highly divergent from species to species. Said epitopes are more preferably selected from fragments of the nSMase3 of embodiment (1), most preferably selected from fragments of the native nSMase3 sequence. In turn, said fragments of the native nSMase3 are preferably fragments of SEQ ID NO:3 to 9. In one preferred aspect of said turn, the antibody of embodiment (10) is raised against one or more of the fragments represented by SEQ ID Nos:17 to 24, which are highly conserved regions in nSMase3s from different species. Said antibody is therefore specific for a set of several nSMase3s. In another preferred aspect of said turn, the antibody is raised against a species-specific nSMase3 fragment, especially a fragment which is not represented by SEQ ID Nos:17 to 24, and is therefore strictly species specific.

The pharmaceutical or diagnostic composition of embodiment (13) may further comprise at least a pharmaceutically or diagnostically acceptable carrier.

There are quite a few diseases, disorders and health conditions which may be connected to changes, i.e. increases or decreases, in the expression and/or activity of nSMase3. These are heart muscle diseases, prominently DCM, virus infections, including VSV, other rhabdoviridae, reovirus, influenza virus, rhinovirus, hepatitis A and C, Ebola virus, SARS coronavirus, HIV, etc. infections, and male reduced fertility or infertility which may be connected with lack of nSMase3 (decrease); and other heart muscle diseases, prominently hypertrophic cardiomyopathy, cramps (including tetanus), and tremor (including Parkinson's disease) which may be connected with overexpression and/or increased activity of nSMase3 (increase). Furthermore, the use of nSMase3 inhibitors as contraceptive in men and male animals is contemplated in present invention.

In the method for diagnosing and/or monitoring a disorder characterized by changes in the expression of an nSMase3 according to embodiment (16), the detection step (b) is preferably performed using immunological or molecular biology methods. If immunological methods are used, one antibody of choice is the antibody of embodiment (10). Furthermore, the activity of nSMase3 can be detected by any method feasible to detect nSMase activity, preferably by a method implying the measurement of enzymatic SM hydrolysis.

The cell, tissue or non-human organism of embodiment (18) is lacking a part or the total of its native nSMase3 content. This is preferably due to treatment of the cell, tissue or organism with an agent suppressing or reducing nSMase3 expression or interfering with the nSMase3 coding gene, including transformation or transfection with heterologous DNA, RNAi, gene targeting including conditional and inducible gene knock out, chemical and radiation mutagenesis. In case of non-human animals or tissue therefrom, the nSMase3 deficiency is preferably generated by constitutive or inducible RNAi or by transgene or conditional gene knockout.

Said cell, tissue or non-human organism may be used as a model for diseases connected with a nSMase3 deficiency (embodiment 19 (i)), preferably for dilated cardiomyopathy and infertility in men or male animals. It may further be used for production of nSMase3 antibodies, preferably of monoclonal antibodies. Said antibodies may be used for modulation of nSMase3 activity in vitro or in vivo.

The medicament of embodiment (20) is preferably used for the treatment of hypertrophic cardiomyopathy, tetanus, muscle cramps and tremor including Parkinson's disease. The treatment of Parkinson's disease is especially preferred. The agent reducing the abundance of nSMase3 in an organism in the use (20) is selected from RNAi agents against nSMase3 including the shRNA and siRNA of embodiment (11) and the vector of embodiment (12), antibodies including the antibody of claim (10) and chemicals inhibiting nSMase3 expression and/or activity.

The effective dose in the methods of embodiments (21) and (22) is dependent on several parameters, inter alia the sex of the patient, his size, age and health condition. Thus, it has to be determined individually from case to case by the responsible physician.

The method of embodiment (23) uses RNAi, a tool for inhibition of gene expression (Fire, A. et al., Nature 391, 806-811 (1998)). It is based on the introduction of double stranded RNA (dsRNA) molecules into cells, whereby one strand is complementary to the coding region of a target gene. Through pairing of the specific mRNA with the introduced RNA molecule, the mRNA is degraded by a cellular mechanism. In mammalian cells, short (<30 bp) interfering RNAs (siRNA) are used in RNAi, as they circumvent the interferon response to long dsRNA (Elbashir, S. M. et al., Nature 411, 494-498 (2001)). Injection of siRNA into the tail vein of mice leads to gene inhibition which is restricted to specific organs and persists only a few days (McCaffrey, A. P. et al., Nature 418, 38-39 (2002); Lewis, D. H. et al., Nature Genet. 32, 107-108 (2002)). A further improvement of the siRNA technology is based on the intracellular transcription of the two complementary siRNA strands using separate expression units both under the control of the U6 promoter (Lee, N. S. et al., J. Nat. Biotechnol. 20(5):500-5 (2002); Du, Q. et al., Biochem. Biophys. Res. Commun. 325(1):243-9 (2004); Miyagishi, M. and Taira, K., Methods Mol. Biol.; 252:483-91 (2004)) or expression of short hairpin RNA (shRNA) molecules by a single transcription unit under the control of the U6 or H1 promoter (Brummelkamp, T. R. et al., Science 296, 550-553 (2002); Paddison, P. J. et al, Genes Dev. 16, 948-958 (2002); Yu, J. Y. et al., Proc. Natl. Acad. Sci. USA 99, 6047-6052 (2002); Sui, G. et al., Proc. Natl. Acad. Sci. USA 99, 5515-5520 (2002); Paul, C. P. et al., Nature Biotechnol. 20, 505-508 (2002); Xia, H. et al., Nat. Biotechnol. 10, 1006-10 (2002); Jacque, J. M. et al., Nature 418(6896):435-8 (2002)).

Thus, shRNA and siRNA against nSMase3 and vectors encoding said RNAs are aspects of present invention (embodiments (11) and (12)).

In present invention, the RNAi target gene in embodiment (23) is the native gene coding for nSMase3. Preferred siRNAs are RNAs of a minimum length of 15 nt and a maximum length of 30 nt. Preferred shRNAs contain a sense and an antisense RNA strand which are connected by a loop; sense and antisense strand each have a minimum length of 15 nt and a maximum length of 30 nt, the loop has a minimum length of 0 nt, preferably of 5 nt, and a maximum length of 15 nt. The most preferred shRNA in the context of present invention is the shRNA having SEQ ID NO:30 or 31. Said siRNAs and shRNAs are complementary to coding regions of the nSMase3 gene, more preferably RNAs which are complementary to regions of the mRNA corresponding to any one of SEQ ID Nos:3 to 9. Said siRNAs and shRNAs may contain a few non-homologous bases, preferably at their ends, or—in case of shRNA—as a loop connecting the sense and antisense strand. However, more preferably the siRNAs do not contain any non-homologous bases. Most siRNA expression vectors are based on polymerase III dependent (Pol III) promoters (U6 or H1) that allow the production of transcripts carrying only a few non-homologous bases at their 3′ ends. It has been shown that the presence of non-homologous RNA at the ends of the shRNA stretches lower the efficiency of RNAi mediated gene silencing (Xia, H. et al., Nat. Biotechnol. 10, 1006-10 (2002)).

A preferred aspect of the method (23) implies the use of shRNA as RNAi agent, more preferably the shRNA as exemplified in example 12, including the vectors and methods used therein.

In the cells, tissue, tissue culture or animals which are the result of the method of embodiment (23), the nSMase3 activity is downregulated or even completely suppressed (compare example 12). Therefore, said cells etc. are ideal model systems for the method of embodiment (8) if they are used as described in embodiment (19(i)).

In the following, the properties of the nSMase3 of present invention are exemplified on the human nSMase3 of SEQ ID NO:3. These properties, however, also apply to other nSMase3s of present invention, especially to vertebrate and even more to mammalian nSMase3s.

The human nSMase3 of present invention was biochemically characterized by overexpression in a yeast strain, JK9-3dΔIsc1p, lacking intrinsic SMase activity. Like nSMase2, nSMase3 is Mg²⁺ dependent, shows enhanced activity in the presence of phosphatidylserine, and is inhibited by scyphostatin. NSMase3 is ubiquitously expressed as a 4.6 kb mRNA species. Tumor necrosis factor (TNF) stimulates rapid activation of nSMase3 in MCF7 cells with peak activity at 1.5 min. nSMase3 lacks an N-terminal signal peptide yet contains a 23 amino acid transmembrane domain close to the C-terminus, which is indicative for the family of C-tail-anchored integral membrane proteins. Cellular localization studies of nSMase3, expressed as green fluorescent protein fusion protein, demonstrated colocalization with markers of the ER. These results suggest that sphingomyelin metabolism regulated by nSMase3 is at the receiving end of TNF signaling.

Recently, a Mg²⁺ ion dependent, 97 kDa nSMase from bovine brain was purified to homogeneity and characterized (Bernardo, K. et al., J. Biol. Chem. 275:7641-7647 (2000)). Amino acid sequencing of tryptic peptides showed no apparent homology to nSMase1 or nSMase2, nor to any known bacterial SMase. By databank searches a human protein with sequence homologies to one of the bovine nSMase peptides was identified. This human homolog was designated as nSMase3 (SMPD4). It functions as a nSMase in cells. Its gene is a structural gene for neutral sphingomyelinase and is located on chromosome 21 at locus 2q21. Furthermore, nSMase3 is activated by TNF and colocalizes with ER markers.

The nSMase3 according to embodiment (1) of the invention is evolutionary highly conserved allowing identification of homologous proteins down to the level of Bilateria (FIG. 1B). This is in contrast to the known nSMase2 that appears to be conserved down only to the level of mammals (Hofmann, K. et al., Proc. Natl. Acad. Sci. USA 97:5895-5900 (2000); NCBI Homologene). The gene locus 2q21 is not associated with known mutations in mice so far. The phenotypic data of deletion mutants of C. elegans produced by large-scale RNA interference techniques are contradictory and range from lethality to absence of any obvious phenotype (Fraser, A. G. et al., Nature 408:325-330 (2000); Piano, F. et al., Curr. Biol. 12:1959-1964 (2002); Maeda, I. et al., Curr. Biol. 11:171-176 (2001)). At least two cardiovascular diseases are associated with the chromosomal locus 2q21—dilated cardiomyopathy (CMD 1H) and susceptibility to premature coronary heart disease (Jung, M. et al., Am. J. Hum. Genet. 65:1068-1077 (1999); Pajukanta, P. et al., Am. J. Hum. Genet. 67:1481-1493 (2000)). The high expression level of the nSMase3 gene (Smpd4) in heart muscle suggests a role of this gene in the pathogenesis of cardiac diseases.

According to the NCBI SNP database (http://www.ncbi.nlm.nih.gov/entrez/query.fcgi?db=Snp), the genomic region of nSMase3 shows a high degree of variability: 25 single nucleotide polymorphisms are described, 10 of them may affect mRNA sequence including four nonsynonymous mutations.

Although no significant sequence homology to nSMase1 or nSMase2 was discerned, nSMase3 displayed biochemical properties similar to nSMase2. When analyzed in yeast cells, nSMase3 like nSMase2 showed dependency for Mg²⁺ and Mn²⁺ ions. In contrast to nSMase2, Mg²⁺ or Mn²⁺ concentrations above 6 mM markedly suppressed nSMase3. As for nSMase2, Ca²⁺, copper or zinc did not support nSMase3 activity. Furthermore, when directly compared in yeast cells, nSMase3 and nSMase2 were similarly stimulated by PS, and both nSMase2 and nSMase3 were equally sensitive to inhibition by scyphostatin.

Increased nSMase activity in mammalian cells can be brought about by cDNAs encoding either a nSMase or a co-factor required for nSMase function. To establish that a structural gene encodes nSMase3, nSMase3 was expressed in inositol phosphosphingolipid phospholipase C (Isc1p)-deficient JK9-3dΔIsc1p yeast cells (Example 3, FIG. 4b). The deletion of Isc1p removes residual endogenous SMase activity and previously allowed Hannun and coworkers to unequivocally define smpd3 (nSMase2) as a gene coding for a bona fide neutral SMase (Marchesini, N. et al., 3. Biol. Chem. 278:13775-13783 (2003)). Similarly, overexpression of nSMase3 in JK9-3d cells established robust nSMase activity in the microsomal fraction, providing pertinent evidence that nSMase3 is a structural enzyme with neutral SMase activity (Example 3, FIG. 4b).

The subcellular topology of nSMase3 expression merits some mention of its possible role in sphingolipid metabolism. The lack of an N-terminal leader signal sequence and its transmembrane domain close to the C-terminus define nSMase3 as a C-tail-anchored integral membrane protein. Notably, nSMase1 also lacks an N-terminal signal sequence, yet its two transmembrane regions are followed by 50 amino acid residues (Tomiuk, S. et al., Proc. Natl. Acad. Sci. USA 95:3638-3643 (1998)). Considering that only 40 amino acids of the nascent chain are sequestered within the eukaryotic large ribosomal subunit (Blobel, G. and Sabatini, D. D., J. Cell Biol. 45:130-145 (1970)), the transmembrane domains of nSMase1 may interact with the signal recognition particle (SRP) as long as they are part of the nascent polypeptide chain. Thus, nSMase1 is probably delivered to the ER by the SRP-dependent pathway. In contrast, like other C-tail-anchored proteins, nSMase3 possesses a hydrophobic membrane insertion sequence at the C-terminus such that it only can emerge from the ribosome after translation is terminated. Although the mechanisms determining the selection of the target membrane have not yet been identified, the principal sites of tail-anchored proteins are the ER and the mitochondrial outer membrane (Borgese, N. et al., J. Cell Biol. 161:1013-1019 (2003); Kutay, U. et al., Embo J. 14:217-223 (1995)). Indeed, nSMase3 showed an ER distribution pattern when N-terminally fused with GFP (example 10, FIG. 7a). It is worth emphasizing that nSMase2 contains two hydrophobic domains in its N-terminal portion (Hofmann, K. et al., Proc. Natl. Acad. Sci. USA 97:5895-5900 (2000)). Thus, nSMase3 distinguishes itself from nSMase1 and nSMase2 by its C-tail anchoring and membrane targeting.

Since SM is synthesized at the Golgi apparatus, the question arises whether the ER is the final destination of nSMase3. The ER localization of nSMase3 has been demonstrated with a GFP fusion protein. Although the retention in the ER is mostly transmembrane and luminal domain-dependent as has been demonstrated with cytochrome b5 (Honsho, M. et al., J. Biol. Chem. 273:20860-20866 (1998)), the N-terminal fusion to a 40 kDa protein such as GFP might alter the physiologic localization of nSMase3. More information about the precise localization of nSMase3 can be expected from stainings of endogenous nSMase3 by specific antibodies, e.g. the antibodies of embodiment (10) of the invention. Thus, nSMase3 may also be localized downstream the secretory pathway, that is, in the Golgi or in the plasma membrane.

A particular feature of the ER membrane is its capacity to support rapid, bidirectional transport of phospholipids across the bilayer (reviewed in Pomorski, T. et al., Semin Cell Dev. Biol. 12:139-148 (2001)). The location and topology of sphingolipid synthesis is rather complex with different reactions occurring in either the ER or Golgi apparatus and with distinct topologies. It is important to note that the active sites of some of these enzymes face the cytoplasm, others face the lumen, which implies transbilayer movement of sphingolipids during the biosynthetic process. The initial steps of sphingolipid synthesis take place at the cytoplasmic leaflet of the ER but later steps such as the conversion of ceramide to sphingomyelin take place for the most part at the luminal leaflet of the Golgi apparatus (for review Futerman, A. H. and Riezman, H., Trends Cell Biol. 15:312-318 (2005)). Thus, ceramide needs to be translocated to the lumen for SM synthesis. While transbilayer movements of glycosylceramides and other sphingolipids require the action of flippases, ceramide may spontaneously flip-flop in lipid bilayers. Thus, one possible physiologic action of nSMase3 would be to hydrolyse SM at the cytoplasmic leaflet of the ER membrane to allow for transbilayer movement of ceramide and subsequent conversion to SM in the lumen of the Golgi apparatus. In this scenario, nSMase3 would be instrumental for the generation and maintenance of the asymmetrical distribution of SM in eukaryotic membranes.

The present invention is described in more detail by reference to the following examples. It should be understood that these examples are for illustrative purpose only and are not to be construed as limiting the invention.

### EXAMPLES

In the following examples, standard techniques of recombinant DNA technology were used that were described in various publications, e.g. Sambrook et al. (1989), Molecular Cloning: A Laboratory Manual, Cold Spring Harbor Laboratory, or Ausubel et al. (1987), Current Protocols in Molecular Biology 1987-1988, Wiley Interscience. Unless otherwise indicated, all enzymes, reagents and kits were used according to the manufacturers' instructions.

### Example 1

### Cell Culture, Media, Cells and Plasmids

MCF7 cells were cultured at 37° C. in RPMI 1640 (Biochrom, Berlin, Germany) supplemented with 10% fetal bovine serum, 2 mM L-glutamine, 100 μg/ml streptomycin and 100 U/ml penicillin (Biochrom) in a humidified 5% CO₂ incubator. HEK293 and HeLa cells were cultured at 37° C. in DMEM (Biochrom, Berlin, Germany) supplemented with 10% fetal bovine serum, 2 mM L-glutamine, 100 μg/ml streptomycin and 100 U/ml penicillin (Biochrom) in a humidified 5% CO₂ incubator. All cells were obtained from the American Type Culture Collection.

Yeast Strain and Culture Media: The Saccharomyces cerevisiae strain JK9-3dΔIsc1p deficient for the Isc1p gene (Sawai, H. et al., J. Biol. Chem. 275:39793-39798 (2000)) was kindly provided by Yusuf Hannun (Charleston, S.C.). Yeast extracts and peptone were from Difco (BD, Heidelberg, Germany). Synthetic minimal medium (SD), SD/Gal, and Ura dropout supplement were purchased from Clontech (BD, Heidelberg, Germany). Scyphostatin was a kind gift of Dr. T. Ogita and F. Nara, Sankyo Co. LTD, Tokyo, Japan.

Plasmids containing full-length coding sequences of human nSMase3 as identified by homology search (compare Example 2) (clone DKFZp434A1711Q) and nSMase2 (clone IRALp962J187Q) were isolated from clones obtained from “RZPD Deutsches Ressourcenzentrum für Genomforschung” (https://www.rzpd.de) and reamplified by PCR for subcloning.

### Example 2

### Sequence Identification and Analysis in Silico

Homology search using the bovine peptide KGLPYLEQLFR (SEQ ID NO:16) against protein sequences derived from a draft of the human genome project was performed using standalone blastp program (Altschul, S. F. et al., Nucleic Acids Res. 25:3389-3402 (1997)). Protein sequence was analyzed for conserved patterns or motifs by search against Prosite (http://ca.expasy.org/prosite/) or Pfam database (http://www.sanger.ac.uk/Software/Pfam/search.shtml), respectively. Prediction of transmembrane region was performed using the TMHMM Algorithm (http://www.cbs.dtu.dk/services/TMHMM/). The ClustalW algorithm was employed for protein sequence alignment.

### Example 3

### Expression of nSMase3 in Yeast

The pYES2 yeast expression vector containing a galactose-inducible promoter was purchased from Invitrogen (Karlsruhe, Germany). cDNA of the human nSMase3 was generated by PCR, using a plasmid isolated from the clone DKFZp434A1711Q as a template and Pfu as proofreading polymerase (Promega, Mannheim, Germany). The following PCR primers were used to introduce EcoRI- and SalI-endonuclease restriction sites to facilitate subcloning:

| | |
|---|---|
| forward | |
| 5′- GGAATTCTGATGACGACTTTCGGCG-3′, | (SEQ ID NO: 25) |
| reverse | |
| 5′- GTCGACGTCAGGGCTGGTGCAGCTT-3′. | (SEQ ID NO: 26) |

YES2 and the PCR product were digested by EcoRI and SalI for 2 h at 37° C. pYes2 was additionally dephosphorylated by addition of 1 U calf intestine alkaline phosphatase (Fermentas, St. Leon-Rot, Germany) for 30 min at 37° C. Fragments were resolved by agarose gel electrophoresis, visualized by ethidium bromide staining and excised from the gel. DNA was recovered from the gel blocks by QiaQuick Gel Extraction Kit (Qiagen, Hilden, Germany). Fragments were ligated by T4 DNA ligase (Fermentas) according to manufacturer's instructions. 2 μl of ligation reaction were transformed into competent E. coli XL1-blue cells.

Human nSMase2 was cloned in a similar way using a plasmid isolated from IRALp962J187Q as template, XhoI and BamHI restriction sites and the following primers:

| | |
|---|---|
| forward | |
| 5′- GGGATCCATGGTTTTGTACACGACC-3′, | (SEQ ID NO: 27) |
| reverse | |
| 5′-CCTCGAGCTATGCCTCCTCCTCCC-3′. | (SEQ ID NO: 28) |

Said cloned human nSMase2 served as a control.

The sequences of the resulting plasmids (SEQ ID NO:13 for nSMase2 and SEQ ID NO:12 for nSMase3) were confirmed by sequencing. Plasmids were transfected into yeast JK9-3dΔIsc1p cells as described by Marchesini et al. (Sawai, H. et al., J. Biol. Chem. 275:39793-39798 (2000)), and the expression of nSMase3 was induced by incubating the cells in synthetic complete Ura medium containing 2% galactose overnight.

### Example 4

### Preparation of Yeast Cell Lysates

Yeast cells were suspended in buffer containing 25 mM Tris (pH 7.4), 5 mM EDTA, 1 mM phenylmethylsulfonyl fluoride, and 4 μg/ml each chymostatin, leupeptin, antipain, and pepstatin A. Cells were disrupted with glass beads as described (Sawai, H. et al., J. Biol. Chem. 275:39793-39798 (2000)). Glass beads and cell debris were removed by centrifugation at 2,000×g for 10 min, and the supernatant was centrifuged at 100,000×g to obtain the microsomal and cytosolic fractions. For the studies on cation and lipid effects, and on substrate requirements of nSMase3, membranes were delipidated by incubation in lysis buffer in the presence of 1% Triton®X-100 and incubated at 4° C. for 1 h. The suspension was centrifuged at 100,000×g for 90 min, and the supernatant was used for enzymatic determinations. Protein concentration was determined using a Bio-Rad protein assay reagent.

### Example 5

### Overexpression of nSMase3 in Mammalian Cells

The enzymatic characterization of nSMase3 was assessed by transient expression in HEK293 and MCF7 (FIG. 3). cDNA constructs (see below) were transfected into MCF7 or HEK293 cells using the Ca²⁺-phosphate precipitation method (Sambrook, J. and Russel, D. W., Molecular cloning: a laboratory manual, 3rd Ed., Cold Spring Harbor Laboratory Press, NY (2001)). Briefly, 1.5*10⁶ HEK293 or 1*10⁶ MCF7 cells were seeded 24 h prior to transfection on 10 cm culture plates (Falcon Primaria, BD). 5-10 μg of plasmid DNA were used for a single transfection. 0.5 ml of DNA solution in 250 mM CaCl₂ was added dropwise to the equal amount of 2×HeBS (0.28 M NaCl, 0.05 M HEPES, 1.5 mM Na₂HPO₄) and vigorously vortexed. The transfection mixture was carefully distributed over the cell plate. For selection of stable transfectants, 800 μg/ml G418 (Invitrogen) was added to the medium. Independent colonies were picked and cultured in separate wells, and three clones from single cells expressing the highest nSMase activity in vitro were used as nSMase3 overexpressors, e.g. in example 9 (FIG. 6A, B).

293 or MCF7 cells were transiently transfected with increasing doses of an expression plasmid, pRK-nSMase3 (SEQ ID NO:10) encoding full-length human nSMase3. 36 h after transfection, membrane fractions were assayed for nSMase activity. Enzymatic assays showed a dose-dependent increase of nSMase activity (FIG. 3A,B). Transfection of nSMase3 into MCF7 cells resulted in a decrease in SM mass, which was comparable to that obtained with nSMase2 transfectants (transfected with pRK-nSMase2, SEQ ID NO:11) used as control (FIG. 3C). It should be noted that combined DAG kinase/TLC analysis revealed ceramide generation by nSMase3 transfected MCF7 cells, which was hardly detectable with untransfected MCF7 cells (FIG. 3D), indicating that parental MCF7 cells express low level, if any, endogenous nSMase activity.

### Example 6

### Assays and Detection Methods

A) Assay for nSMase: The micellar SMase assay using exogenous radiolabeled SM was described by Wiegmann et al. (Wiegmann, K. et al., Cell 78:1005-1015 (1994)). To measure neutral SMase, cultured cells were harvested by standard trypsin-EDTA treatment and pelleted by centrifugation (300×g, 15 min). The pellets were dissolved in a buffer containing 20 mM Hepes (pH 7.4), 10 mM MgCl₂, 2 mM EDTA, 5 mM DTT, 0.1 mM Na₃VO₄, 0.1 mM Na₂MoO₄, 30 mM p-nitrophenylphosphate, 10 mM β-glycerophosphate, 750 μM ATP, 1 mM PMSF, 10 μM leupeptin, 10 μM pepstatin, 0.5% Chaps. After 15 min at 4° C., cells were homogenized by repeated squeezing of the cells through an 18-gauge needle. Nuclei and cell debris were removed by a low speed centrifugation (800×g). The protein concentration in supernatants containing the cytosolic and membrane fractions was measured using a protein assay (Pierce, Hamburg, Germany). 50 μg of protein were incubated for 2 h at 37° C. in a buffer containing 20 mM Hepes, 1 mM MgCl₂ (pH 7.4) and 2.25 μl of [N-methyl-¹⁴C]SM. Phosphorylcholine was then extracted with 800 μl of chloroform:methanol 2:1 (v/v) and 250 μl of H₂O. Radioactive phosphorylcholine produced from [¹⁴C]SM was identified by counting 200 μl of the aqueous phase by scintillation counting.

B) Sphingomyelin Measurement: Cells were seeded at 0.3×10⁶ cells/10-cm-dish in 8 ml of complete growth medium. The next day, the cells were labeled with [methyl-³H]choline chloride (1 μCi/ml final concentration in 10 ml of growth medium/plate) for 48 h. Cells were then chased with 10 ml complete medium. At the indicated time points, the medium from each plate was collected, and the cells were washed once with 2 ml of ice-cold PBS. Cells were scraped off on ice in 2 ml of PBS, and each plate was washed with an additional 2 ml of PBS. Cells and washes were pooled with the medium and centrifuged for 5 min at 2,000×g (4° C.). Lipids were extracted by the method of Bligh and Dyer (Bligh, E. G. and Dyer, W. J., Can. J. Biochem. Physiol. 37:911-917 (1959)), and aliquots of 250 μl were used for phosphorus and SM determination as previously described (Wiegmann, K. et al., Cell 78:1005-1015 (1994)).

C) Ceramide Measurement: The generation of the neutral lipid cleavage product of SMases, ceramide, was detected by the DAG-kinase assay combined with TLC analysis. Briefly, MCF7 cells were washed two times in ice-cold PBS. Cultured cells were harvested by standard trypsin-EDTA treatment and pelleted by centrifugation (300×g, 15 min). The pellets were resuspended in 1.825 ml H₂O and the supernatants were transferred into glass tubes. Subsequently, 6 ml CHCl₃/MeOH/1N HCl (100/100/1 v/v/v) were added. Tubes were sonicated for 5 min in a water bath sonicator and then centrifuged for 10 min at 6000×g. The lower organic phase was dried down under nitrogen. The dry samples were resuspended in 2 ml 0.1 N KOH and incubated for 1 h at 37° C. (alkaline hydrolysis). 6 ml CHCl₃/MeOH/1N HCl (100/100/1 v/v/v), 1 ml CHCl₃ and 2.25 ml water were added. After vortexing the samples were centrifuged for 10 min at 6,000×g. The lower organic phase was dried down under nitrogen and resuspended in 100 μl CHCl₃ followed by transfer into Eppendorf tubes. From this point on, the DAG-kinase assay kit protocol was followed (Amersham Biosciences). For thin-layer chromatography, plates were pre-run in a solvent system composed of methanol/chloroform (1:1). Plates were removed from the tank and air-dried. TLC-chambers were pre-equilibrated for 1 h at room temperature with the solvent system cloroform:methanol:acidic acid (65:15:5, v/v/v). Dried samples were resuspended in 20 μl of chloroform:methanol (95:5, v/v). Samples were streaked onto a 10×10 cm silica gel thin layer plate. Plates were placed in a paper-lined TLC developing tank and the solvent was allowed to migrate to the top of the plate. Plates were removed from the tank, air-dried and exposed to Kodak XAR-films at room temperature.

D) Immunofluorescence: Cells were grown on coverslips and transfected with 1 μg nSMase3 DNA constructs using ExGen 500 in vitro transfection reagent (Fermentas, St. Leon-Rot, Germany) and incubated for 24 h. Cells were washed twice with cold PBS, then fixed with 3% paraformaldehyde for 20 min, permeabilized with 0.1% saponin in PBS for 10 min, and blocked with 3% BSA, 0.005% sodium azide, 0.2% teleostean gelatin and 0.1% saponin in PBS for 30 min. For immunostaining, cells were incubated with rat anti-KDEL antibody (kindly provided by Dr. G. Butcher, Cambridge) for 1 h, washed with 0.1% saponin in PBS and then incubated for 30 min with Cy3-conjugated goat anti-rat IgG (Dianova, Hamburg, Germany) as secondary reagent. Nuclei were counterstained with Hoechst 33258 and examined using a confocal fluorescence microscope.

### Example 7

### Identification of a Human Homolog to Bovine nSMase

The purification to homogeneity and biochemical characterization of a Mg²⁺-dependent nSMase from bovine brain was reported previously (Bernardo, K. et al., J. Biol. Chem. 275:7641-7647 (2000)). Amino acid sequencing of tryptic peptides of bovine nSMase showed no homologies to any known nSMases. Allowing for mismatches due to possible species-specific primary sequences, extensive sequence database searches revealed a homology to the human hypothetical protein FLJ20297 (synonyms KIAA1418 and LOC55627, accession number BAA92656). The predicted mRNA contains an ORF that encodes a protein of 866 amino acids (aa), resulting in a predicted molecular mass of 97.8 kDa, which matched the molecular mass of the nSMase previously purified from bovine brain.

An alignment of the novel nSMase3 with nSMase1 and nSMase2 is shown in FIG. 1A. Apparently, nSMase3 lacks an N-terminal signal peptide. In addition, a C-terminal hydrophobic region (aa 828-850) representing a putative transmembrane domain, is followed by only 16 amino acids. The lack of an N-terminal leader sequence combined with a hydrophobic transmembrane domain close to the C-terminus defines the group of tail-anchored integral membrane proteins, whose entire functional N-terminal portion faces the cytosol (Borgese, N. et al., J. Cell Biol. 161:1013-1019 (2003)). Other than that no statistically significant patterns or motifs were recognized.

In particular, a possible ER membrane protein motif (KLHQ) is located near the C-terminus (aa 862-865) that, however, does not match with the minimal ER retention consensus sequences (KDEL, KKXX or KXKXX). PSORT II analysis, (http://psort.nibb.ac.jp) predicts that the subcellular location of this protein is most likely the endoplasmic reticulum (55.6%), followed by mitochondria (22.2%), plasma membrane (11.1%) and vesicles of the secretory system (11.1%) (Nakai, K. and Horton, P., Trends Biochem. Sci. 24:34-36 (1999)). Strikingly, the alignment with mammalian nSMase1 and nSMase2 showed no apparent homologies. The database search revealed proteins closely related to nSMase3 in mouse, dog, chicken, zebrafish, drosophila and C. elegans (FIG. 1B).

The gene for nSMase3 is encoded on chromosome 2, chromosomal locus 2q21.1. Although the predicted gene is comprised of about 31 alternative exons and may result in 12 different splice variants, we found that nSMase3 mRNA was ubiquitously expressed as single 4,600 nt mRNA species, with the exception of leukocytes where two signals were visible. The greatest abundance of nSMase3 mRNA was observed in striated muscle, heart muscle an testis (FIG. 2). It should be noted, that the current version of the Genebank database defines two shorter isoforms of FLJ20297 mRNA as a reference sequence. This reflects alternate splicing events that were hypothesized to occur on theoretical grounds and should result in mRNAs with 3,600 and 3,300 nucleotides, respectively. The 4.6 kb size of nSMase3 mRNA, however, suggests abundant expression of full length cDNA corresponding to the cDNA clone with accession number AB037839.

### Example 8

### Characterization of nSMase3 Enzymatic Activity

Increased nSMase activity in mammalian cells can be brought about by cDNAs encoding either a nSMase or a cofactor required for nSMase function. To establish that nSMase3 is encoded by a structural gene for neutral sphingomyelinase, nSMase3 was expressed in JK9-3dΔIsc1 yeast cells. This strain of S. cerevisiae lacks the isc1 locus encoding an inositol phosphosphingolipid phospholipase C (Isc1p) that exerts SMase activity (Sawai, H. et al., J. Biol. Chem. 275:39793-39798 (2000)). The deletion of Isc1p removes any residual endogenous SMase activity and thus allows detection of the nSMase enzyme itself (Marchesini, N. et al., J. Biol. Chem. 278:13775-13783 (2003)). Like nSMase2, overexpression of nSMase3 in JK9-3dΔIsc1p cells established robust nSMase activity in the microsomal fraction prepared by the method of example 4 (FIG. 4A), indicating that nSMase3 is a structural nSMase. Kinetic analysis of nSMase3 in the presence of PS revealed for SM a Kₘ of 18.71 μM and a Vₘₐₓ of 1.67 pmol/μg/h (FIG. 4B). MgCl₂ and MnCl₂ at 5 mM stimulated nSMase3, while CaCl₂, CUCl₂ or ZnCl₂ did not change nSMase3 activity (FIG. 5A). PS stimulated nSMase3, while other phospholipids had only slight effects (FIG. 5B). As shown for the purified bovine nSMase (16), nSMase3 was inhibitable by scyphostatin in a dose-dependent manner (FIG. 5C).

### Example 9

### TNF-Induced Activation of nSMase3

nSMase can be stimulated by ligand cell surface receptor interactions including the TNF/TNF receptor system. In order to test whether nSMase3 is responsive to TNF, MCF7 cells stably transfected with nSMase3 were employed and compared to parental MCF7 cells that express little if any nSMase activity (FIG. 3B,C). Stable expression of nSMase3 in MCF7 cells revealed cell clones with markedly increased nSMase activity (FIG. 6A). TNF stimulation according to Wiegmann, K. et al., Cell 78:1005-1015 (1994) of MCF7-clone23 overexpressors resulted in a 250% increase of nSMase activity within 1.5 min (FIG. 6B). In contrast, TNF did not stimulate nSMase activity in mock transfected MCF7 cells, indicating that the increments of nSMase activity observed in TNF-treated cells can be attributed to the transfected nSMase3.

### Example 10

### Subcellular Localization of nSMase3

In order to address the subcellular localization of nSMase3, HeLa cells were transiently transfected with DNA constructs expressing GFP-nSMase3 (N-terminally fused GFP). The fusion of GFP to the N-terminus was chosen in consideration of the primary structure of nSMase3 that predicted membrane anchoring through the C-terminal hydrophobic domain. The open reading frame of human nSMase3 (and nSMase2) was amplified by PCR using nSMase primers containing XhoI/EcoRI restriction sites as described in Example 3. PCR products were subjected to restriction digestion with corresponding restriction enzymes and the digested DNA fragments were ligated to digested pEGFP-N3 (nSMase3 fused to the N-terminus of EGFP, pGFP-nSMase3; SEQ ID NO: 15) (Clontech, Germany) expression vectors.

As shown in FIG. 7A, immunofluorescence analysis of GFP-nSMase3 expression pattern revealed intensive perinuclear and diffused reticular structures significantly overlapping with ER visualized by an antibody specific for KDEL (carboxy-terminal sequence of luminal ER proteins). This confirms the predicted localization of nSMase3 as an ER membrane protein. Fusion of GFP to the N-terminus did not impair the nSMase enzymatic activity (FIG. 7B).

### Example 11

### Infection of Cardiomyocytes with AdCGIpnSMase3 in Comparison to AdCGI and Non-infected Controls

Single rat ventricular myocytes were obtained from adult Sprague-Dawley rats (200-250 g) by enzymatic dissociation as previously described (Hoppe et al., PNAS 98:5335-5340 (2001)). Immediately after isolation cells were resuspended in medium 199 (Bio Whittaker, Walkersville, Md.) supplemented with 10% fetal bovine serum and 1× penicillin-streptomycin and plated for primary culture on laminin-coated (Sigma) coverslips. Infection of cardiomyocytes was performed 2 h after plating at a multiplicity of infection (MOI) of 15 to 100 p.f.u. per cell with bicistronic adenoviral vectors encoding EGFP and p97 under the control of a CMV promoter (AdCGIpnSMase3, SEQ ID NO:29). Control cells were infected with an adenovirus encoding the reporter gene EGFP alone (AdCGI, SEQ ID NO:14). Cells were maintained in infection media for 4 h at 37° C., after which it was replaced with culture medium. Field stimulated cell contraction was measured 24 to 32 h after infection. After 24 to 32 h the cells were transferred into Tyrode's solution (NaCl 135 mM, KCl 4.0 mM, MgCl₂ 1.0 mM, NaH₂PO₄ 0.33 mM, HEPES 10.0 mM and Glucose 10.0 mM, pH adjusted to 7.3 with NaOH) with the addition of 2 mmol/l Ca²⁺. The cell suspension was placed into a stimulation chamber mounted on the stage of an inverted confocal laser scanning microscope (Leica Microsystems, Wetzlar, Germany). Field stimulation was performed with an electric field stimulator (Grass Medical Instruments, Massachusetts, USA), at a frequency of 1 Hz, with a voltage of 80 V. Cell contraction was recorded using the microscope in line scan mode at 800 Hz and stored as digital images. During the recording procedure, the cells were superfused with fresh Tyrode's solution containing 2 mM Ca²⁺.

Systolic cell shortening was significantly larger in AdCGIpnSMase3-infected cardio-myocytes (14.04±0.91%, n=40) versus AdCGI-infected controls (8.12±1.68%, p=0.00015, n=22) and non-infected controls (7.81±0.83%, p=0.017, n=33). Diastolic cell length was not significantly different between the three groups: 89.91±2.71 μM (AdCGIpnSMase3-infected cells, n=40) versus 85.46±4.80 μM (AdCGI-infected controls, n=22) versus 91.52±4.02 μM (non-infected controls, n=33).

### Example 12

### Downregulation of nSMase3 Activity by Expression of shRNA

A HeLa cell line deficient in nSMase3 was generated by a stable expression of nSMase3 shRNA employing a lentiviral transfection system (Invitrogen). Complementary oligonucleotides (SEQ ID NOs:30 and 31) comprising a 19 nt specific target sequence derived from the human nSMase3 mRNA, 9 nt loop and 19 nt of antisense target sequence as well as overhangs of BglII and HindIII restriction sites were synthesized by Operon (Cologne, Germany). The oligonucleotide dimers were ligated into the pEntry-sh Vector. pEntry-sh was constructed by ligation of XbaI/KpnI fragments of pSuper (OligoEngine), containing RNA-polymerase promoter H1 (TypIII-RNAPol), into XbaI/KpnI digested pENTR (Invitrogen). The resulting construct was designated pENTRY-shnSMase3 (SEQ ID NO:32). The shRNA expression cassette was subcloned into pLenti6-sh vector using LR recombinase mix II (Invitrogen) according to the manufacturer's instructions, resulting in pLenti-shnSMase3 (SEQ ID NO:33). pLenti6-sh was constructed from pLenti6/V5-DEST (Invitrogen) by partial digesting with KspI and KpnI restriction endonucleases, removal of 3′ protruding termini by T4 DNA polymerase and religation of the vector.

Recombinant lentiviruses were generated according to the manufacturer's instructions: pLenti-shnSMase3 together with three packaging plasmids was transformed into 6×10⁶ HEK-293 FT cells using Lipofectamine-2000 Reagent (Invitrogen). Culture supernatants were collected 24 and 48 h after infection and stored at −70° C. Virus titer was determined according to the Invitrogen protocol using the HT1080 cell line. For generation of the shRNA expressing cell line, virus stock was applied at multiplicity of infection of 1 to the semiconfluent HeLa cells. Transfected cells were selected by presence of 10 μg/ml Blasticidin (PAA) for 10 days and subcloned. The integration of the shRNA expression cassette was verified by PCR. Efficacy of downregulation of nSMase3 mRNA was verified by Northern Blot and enzymatic assay for neutral sphingomyelinase as described in example 6A (FIG. 10). Stable expression of the nSMase3-shRNA resulted in a marked decrease of nSMase activity up to 90%. Expression of control shRNAs, like Bax-shRNA or DFIF-shRNA, which are directed to other proteins, did not affect the sphingomyelinase activity in HeLa cells.

### Example 13

### pH Optimum of nSMase3

In order to verify that nSMase3 is a neutral sphingomyelinase a determination of nSMase3 activity at different pH was performed. Protein extracts from MCF7-nSMase3 overexpressing cells (clone23) were prepared as described in example 6A, using Hepes buffer with different pH values ranged from 3.0 to 11.0. As shown in FIG. 8 only assay conditions at neutral pH (7.2) resulted in detectable sphingomyelinase activity. Higher or lower pH values resulted in the complete inhibition of the enzymatic activity of nSMase3. This data indicated that the nSMase3 enzyme is a true neutral sphingomyelinase.

### Example 14

### HeLa Cells with Reduced nSMase3 Activity are Less Succeptible to Viral Infection

A HeLa cell line rendered deficient in the expression of nSMase by stable expression of shRNA as described in example 12. 3×10³ HeLa cells were seeded in each well of a 96-Well plate. VSV was added to cell culture medium at a multiplicity of infection of 10. Viability of HeLa cells cell was measured 24 h after infection by the XTT Assay (Roche) according to the manufacturer's instructions. VSV induced profound cytotoxicity in infected host cells. Only about 17% of wt Hela and 10% of HeLa cells expressing shRNA against DDIF survived after infection. In contrast, HeLa cells expressing shRNA against nSMase3 were protected from virus-induced cytotoxicity and showed significantly higher survival rate (about 50%). These data suggest that the nSMase establish an antiviral protection in HeLa cells.

1. An isolated neutral sphingomyelinase (nSMase3) having the amino acid sequence represented by one of SEQ ID NOs:3 to 9, or a homologue, functional variant, derivative or fragment thereof which possesses the enzymatic and/or immunologic properties of said nSMase3.

2. The isolated nSMase3 of claim 1, which
(i) consists of the amino acid sequence represented by one of SEQ ID NOs:3 to 9, or is a homologue thereof which possesses the enzymatic and/or immunologic properties of said nSMase3; and/or
(ii) contains an amino acid sequence as represented by SEQ ID NO: 16 or 17, preferably contains the amino acid sequence as represented by SEQ ID NO:23, more preferably contains one or more of the amino acid sequences as represented by SEQ ID NOs: 18 to 24; and/or
(iii) lacks an N-terminal leader signal sequence; and/or
(iv) is a vertebrate nSMase3, preferably a mammalian nSMase3, more preferably a human nSMase3.

3. The isolated nSMase3 of claim 1 or 2, which is the human nSMase3 having SEQ ID NO:3, or which is a homologue, functional variant, derivative or fragment thereof, preferably is the human nSMase3 represented by SEQ ID NO:3, or is a homologue thereof.

4. The isolated nSMase3 of any one of claim 1 to 3, which
(i) contains a transmembrane domain close to the C terminus; and/or
(ii) is Mg²⁺ dependent; and/or
(iii) is activated by TNF; and/or
(iv) has a pH optimum at neutral pH; and/or
(v) colocalizes with ER markers.

5. An isolated nucleic acid sequence encoding the nSMase3 as defined in any one of claims 1 to 4, or a nucleic acid complementary thereto.

6. The isolated nucleic acid sequence of claim 5 which comprises the sequence of SEQ ID NO:1 or 2.

7. A vector comprising the nucleic acid sequence of claim 5 or 6.

8. A cell being transfected or transformed with the vector of claim 7 and/or heterologously comprising the nucleic acid of claim 5 or 6 and/or being capable of heterologously expressing an nSMase3 as defined in any one of claims 1 to 4.

9. A tissue, tissue culture or a non-human transgenic organism comprising cells being transfected or transformed with the vector of claim 7 and/or heterologously comprising a nucleic acid sequence as defined in claims 5 or 6 and/or heterologously expressing a nSMase3 as defined in any one of claims 1 to 4.

10. Use of the cell of claim 8, the non-human transgenic organism, the tissue or tissue culture of claim 9 as a test cell system, test tissue or test model organism in testing candidate active agents for their therapeutic potential in diseases connected with nSMase3, preferably in heart muscle diseases, viral infections, male reduced fertility or infertility, or for their potential as contraceptive in men or male animals.

11. A method for testing candidate active agents for their therapeutic potential in diseases connected with nSMase3, preferably in heart muscle diseases, viral infections, male reduced fertility or infertility, or for their potential as contraceptive in men or male animals, comprising the steps
(a) administering the candidate agent to a culture of the cell of claim 8, a non-human organism, a tissue or tissue culture of claim 9; and
(b) determining the effect of the candidate agent on said cell, non-human organism or tissue.

12. A method for preparing the nSMase3 of any one of claims 1 to 4 which comprises culturing cells as defined in claim 8 and isolating the nSMase3 from the culture.

13. An antibody specific for the nSMase3 as defined in any one of claims 1 to 4.

14. A shRNA or siRNA against nSMase3.

15. The shRNA of claim 14, which comprises the sequence represented by SEQ ID NO:30 and/or 31.

16. A vector encoding the shRNA or siRNA of claim 14 or 15.

17. A pharmaceutical or diagnostic composition comprising one or more of the following: the isolated nSMase3 of anyone of claims 1 to 4, the nucleic acid of claim 5 or 6, the vector of claim 6 or 16, the transformed or transfected cell of claim 8, the shRNA or siRNA of claim 14 or 15, and the antibody of claim 13, and pharmaceutically or diagnostically acceptable salts and derivatives thereof.

18. Use of the isolated nSMase3 of any one of claims 1 to 4, the nucleic acid of claim 5 or 6, the vector of claim 7, and/or pharmaceutically acceptable salts or derivatives thereof for preparing a medicament for prevention or treatment of heart muscle diseases, viral infections, male reduced fertility or infertility.

19. Use of the antibody of claim 13, the shRNA and siRNA of claim 14 or 15, the vector of claim 16, other nSMase3 inhibitors including other RNAi agents, and/or pharmaceutically acceptable salts or derivatives thereof for preparing a medicament (a) for prevention or treatment of diseases connected with increased activity of nSMase3, preferably cramps, tremor, Parkinson's disease and hypertrophic cardiomyopathy, or (b) for contraception in men or male animals, or c) for prevention or treatment of viral infections.

20. A method for diagnosing and/or monitoring a disorder characterized by changes in the expression of an nSMase3 as defined in any one of claims 1 to 4, which method comprises the steps
(a) isolating a biological sample from a subject having or suspected to have said disorder;
(b) detection of the amount and/or activity of said nSMase3 or of the nucleic acid sequence encoding said nSMase3, and/or of mutations present in said nucleic acid sequence in comparison to the native nSMase3 encoding sequence

21. Kit for performing the diagnostic method of claim 20, comprising one or more of the following: the nSMase3 of claims 1 to 4, the nucleic acid of claim 5 or 6, the vector of claim 7, the cell of claim 8, the antibody of claim 13, and diagnostically acceptable salts and derivatives thereof.

22. An isolated cell, a tissue or tissue culture, or a non-human transgenic organism, having a reduced abundance of nSMase3 or being deficient in nSMase3 or in the nSMase3 gene, or comprising a siRNA or shRNA of claim 14 or 15 or a vector of claim 16.

23. The cell, tissue or tissue culture or non-human organism, of claim 22, wherein the reduced abundance of nSMase3 or deficiency in nSMase3 or in the nSMase3 gene is achieved by
(i) RNAI (RNA interference), preferably using a shRNA or siRNA of claim 14 or 15 or a vector of claim 16, or
(ii) gene targeting, including conditional and inducible gene knock out, or
(iii) chemical or radiation mutagenesis.

24. Use of the cell, the non-human organism, the tissue or tissue culture of claim 22 or 23
(i) as a model for a disease connected with a deficiency of nSMAse3; or
(ii) for the generation of antibodies to nSMase3, preferably for the generation of monoclonal antibodies including the antibodies of claim 13, and/or of antibodies capable of modulating nSMase3 activity.

25. Use of agents reducing the abundance of nSMase3 in an organism for preparing a medicament for the treatment of diseases connected with increased heart or skeletal muscle contractility, viral infections, and for reducing the fertility in male patients or male animals.

26. The use of claim 25, wherein
(i) the agent reducing the abundance of nSMase3 in an organism is selected from RNAi agents, preferably a shRNA or siRNA of claim 14 or 15 or a vector of claim 16, to nSMase3, antibodies including the antibodies of claim 13 and chemicals inhibiting nSMase3 expression and/or activity; and/or
(ii) the disease is one or more selected from hypertrophic cardiomyopathy, tetanus, muscle cramps, and tremor including Parkinson's disease.

27. A method for preventing or treating heart muscle diseases, especially DCM, or male reduced fertility or infertility, or viral infections, which method comprises administering to the patient an effective dose of one or more of the following: the nSMase3 of claims 1 to 4, the nucleic acid of claim 5 or 6, the vector of claim 7, and pharmaceutically acceptable salts and derivatives thereof.

28. A method for (a) preventing or treating heart muscle diseases connected with increased activity of nSMase3, preferably hypertrophic cardiomyopathy, or (b) reducing the fertility in a male patient or male animal, or c) preventing or treating viral infections, which method comprises administering to the patient an effective dose of one or more of the following: an nSMase3 inhibitory agent, the antibody of claim 13, the shRNA and siRNA of claim 14 or 15, the vector of claim 16, any other RNAi agent to nSMase3, and pharmaceutically acceptable salts and derivatives thereof.

29. A method for downregulating the expression of nSMase3 in vivo and in vitro by RNA interference (RNAi), preferably by administering to the target cell or organism one or more of the group consisting of the shRNA and siRNA of claim 14 or 15 and the vector of claim 16.
